SEQUENCE LISTING (13 sequences)

SEQ ID NO: 1 - protein, 650 aa, Arabidopsis thaliana
  MISC_FEATURE (1)..(650): At2g04842.1 - threonyl-tRNA synthetase
MASSHSLLFSSSFLSKPSSFTSSLRRFVYLPTRQFWPRQRHGFSTVFAVATEPAISSSGPKKAEPSTVVLPSNESSDKLLKIRHTCAHVMAMAVQKLFPDAKVTIGPWIDNGFYYDFDMEPLTDKDLKRIKKEMDRIISRNLPLLREEVSREEAKKRIMAINEPYKMEILDGIKEEPITVYHIGNEWWDLCAGPHVETTGKINKKAVELESVAGAYWRGDEKRQMLQRIYGTAWESEEQLKAYLHFKEEAKRRDHRRIGQDLDLFSIQDEAGGGLVFWHPKGAIVRNIIEESWKKMHVEHGYDLIYTPHVAKADLWKISGHLDFYRENMYDQMEIEDELYQLRPMNCPYHILLYQRKRQSYRDLPIRVAELGTVYRYELSGSLHGLFRVRGFTQDDAHIFCLEDQIKDEIRGVLDLTEEILSRFGFNKYEVNLSTRPEKSVGGDDIWEKATCALRDALDDKGWSYEVDEGGGAFYGPKIDLKIEDALGRKWQCSTIQVDFNLPQRFDITYVDTNSDKKRPIMIHRAVLGSLERFFGVLIEHYAGDFPLWLSPVQVRVLPVTDNQLEFCKEVSKKLRACGVRAELCHGERLPKLIRNAETQKIPLMAVVGPKEVETGTVTVRSRFGGELGTIPVDDLINKINIAVETRTAL

SEQ ID NO: 2 - DNA, 1953 bp, Arabidopsis thaliana
  misc_feature (1)..(1953): DNA sequence encoding At2g04842.1 (SEQ ID NO1)
atggcctcttctcattctcttctcttttcttcttccttcctctctaaaccctcttctttcacttcttctctgagacggtttgtttaccttcccactcgccaattttggccaaggcagcgacatggtttctctaccgtcttcgccgtcgccaccgagccagctatctccagcagcggtcctaagaaagcagagccatcgacggtcgtgcttcctagtaatgaatcatctgataagcttctcaaaattcgacacacgtgtgcgcatgtaatggcaatggcagttcagaaactcttcccggatgcaaaagtgacgataggtccttggattgataatggtttctactatgattttgatatggagcctttaacagataaagaccttaagaggatcaagaaagagatggatcgtatcattagtcggaatttaccacttttaagagaagaagtatctagagaagaagcgaagaaaaggatcatggcgataaatgagccttataagatggagattttggatggtattaaggaagaacccatcaccgtttatcatatcggtaatgaatggtgggatctttgtgcgggtcctcatgtcgagaccacagggaagataaacaagaaagcagttgaactagaatctgttgctggtgcatactggagaggagatgaaaagaggcaaatgcttcagaggatatacggaacagcttgggagagcgaagagcaactgaaagcatatcttcattttaaagaagaagctaaacgcagggatcatagacgcatcggccaagaccttgatctgttttctatccaggatgaagctggaggagggttagtgttttggcatccgaaaggtgcaattgttagaaacataattgaagaatcgtggaagaaaatgcatgttgaacatggttatgatttgatctatactcctcatgttgccaaagctgacctgtggaagatcagtggtcacttggacttctacagagagaatatgtatgatcaaatggagatcgaggatgagctttatcaactccgaccaatgaattgcccttaccacatcttgctttaccaaagaaagcgtcaatcatatcgagatttgcccataagagttgcagagctcggtacagtttacagatatgagttgtctggtagcttgcacggtctcttccgtgtcagagggttcacacaggacgatgcacacatattttgtttagaggatcagatcaaagatgagatcagaggtgttctagatctcacagaagagatattgtcgaggtttggattcaacaagtatgaggttaacttatcaactagacctgaaaaatcagttggaggtgatgatatatgggagaaggcgacctgtgcccttagagatgctttagatgataagggatggagctatgaggttgacgaaggaggtggcgccttttacggcccaaagatcgaccttaagattgaagatgcattgggtagaaaatggcagtgctcaactattcaggtcgacttcaatcttcctcagagatttgacattacctatgtagacacaaactcagataagaaacgtcctataatgatccacagagcggttttaggatcattagaaagatttttcggtgttctcatagaacactacgcaggggatttcccgttatggctctctcctgttcaagtccgggtcttacctgtcacagacaatcagttggagttttgtaaggaagtgagtaagaaactgagagcttgtggagtgagggctgagttgtgccatggggagagactgcctaagctcataaggaacgcagagacgcagaagatcccgttaatggcggttgttggacccaaagaagtcgagaccggaactgttaccgttagatcgaggtttggaggagagcttggaaccattcctgttgatgacctcatcaacaaaatcaacattgcagttgaaaccagaactgctctgtaa

SEQ ID NO: 3 - protein, 65 aa, Arabidopsis thaliana
  MISC_FEATURE (1)..(65): At_dTP - Dual targeting peptide comprising the first 65 amino acids of SEQ ID NO1
MASSHSLLFSSSFLSKPSSFTSSLRRFVYLPTRQFWPRQRHGFSTVFAVATEPAISSSGPKKAEP

SEQ ID NO: 4 - DNA, 195 bp, Arabidopsis thaliana
  misc_feature (1)..(195): DNA sequence encoding At_dTP (SEQ ID NO3)
atggcctcttctcattctcttctcttttcttcttccttcctctctaaaccctcttctttcacttcttctctgagacggtttgtttaccttcccactcgccaattttggccaaggcagcgacatggtttctctaccgtcttcgccgtcgccaccgagccagctatctccagcagcggtcctaagaaagcagagcca

SEQ ID NO: 5 - DNA, 195 bp, Artificial Sequence
  Synthetic - Soybean codon optimized DNA sequence encoding At_dTP (SEQ ID NO3)
atggcttccagtcattcactgcttttttcttcttctttcctgtcaaaacctagctccttcacttccagccttcgccgttttgtgtatcttcctacaagacaattttggcccaggcaacgtcatggctttagtaccgttttcgccgtcgcaaccgaacccgccatatcatcatccgggccaaagaaagccgaacca

SEQ ID NO: 6 - DNA, 235 bp, Artificial Sequence
  Synthetic - Cloning construct comprising the codon optimized DNA sequence of SEQ ID NO5
ttcgaattcggtctcaccatggcttccagtcattcactgcttttttcttcttctttcctgtcaaaacctagctccttcacttccagccttcgccgttttgtgtatcttcctacaagacaattttggcccaggcaacgtcatggctttagtaccgttttcgccgtcgcaaccgaacccgccatatcatcatccgggccaaagaaagccgaaccaggaatgagagaccaagcttgaa

SEQ ID NO: 7 - protein, 652 aa, Glycine max
  MISC_FEATURE (1)..(652): Glyma.19G191200.1 - Soybean homolog of At2g04842.1 threonyl-tRNA synthetase
MATFAVATTLSTSSIAPLNARRGFGFGFGSFLSPTTKPKRPSITTTLAVATDSSVCFNPNPNPNPNPNQLVLPSNESSEMLLRIRHTSAHVMAMAVQKLYPDAKVTIGPWIENGFYYDFDMEPLTDKDLKRIKKEMDRIIGKNLPLVREEVSRGEAHRRISALNEPYKLEILESIKDDPITIYHIGDEWWDLCAGPHVDSTGHINKKAVELESIAGAYWRGDERKPMLQRIYGTAWENEEQLKAYLHFKEEAKRRDHRRLGQDLDLFSIQDDAGGGLVFWHPKGAMVRHIIEDFWKKIHMKRGYDLLYTPHVAKADLWKISGHLDFYKENMYDQMSVEDELYQLRPMNCPYHILVYKSKLHSYRDFPIRVAELGTVYRYELSGSLHGLFRVRGFTQDDAHIFCLDDQIKDEIRGVLDLTEEILLQFGFEKYEVNLSTRPEKAVGDDDIWEKATSALEDALDDKGWTYQIDDGGGAFYGPKVDIKIEDALGRKWQCSTIQVDFNLPQRFDITYVDSNTEKRRPIMIHRAVLGSLERFFGILIEHYAGDFPLWLSPIQARVLPVTDAQLEYCNVVTNKLKMYGIRAEVCHGERLPKLIRNAEKQKIPLMVVVGSKEVETETVTVRSRFGGELGTMPVDDFIDRIKLGTENPISL

SEQ ID NO: 8 - DNA, 1959 bp, Glycine max
  misc_feature (1)..(1959): DNA sequence encoding Glyma.19G191200.1 (SEQ ID NO7)
atggctaccttcgcggttgcaacaacactttcgacatcttctatcgctccccttaatgcacgacgtggttttggttttggttttggctccttcctttctccaaccacaaaaccaaaacgcccctcaatcaccactaccctcgctgtcgccaccgactcttccgtttgtttcaatcccaaccccaaccctaaccctaatcccaatcagcttgttctcccttccaacgaatcatcggagatgcttctcagaatccgccacacgagtgcgcatgtgatggcaatggctgttcaaaagctttaccctgatgcaaaagtcacaattgggccttggatagaaaatgggttttattatgacttcgacatggagcctttgactgataaagatttgaagagaattaagaaagagatggatcgaattattggaaaaaatttgccacttgtaagagaagaagtttcaagaggcgaagctcatagaagaatatcggctctgaatgaaccatacaagttggagattttggaaagcattaaggatgatcccatcaccatttatcacatcggtgatgagtggtgggacctctgtgctggacctcatgttgattctactggacatatcaataagaaagcagttgaacttgagtctattgctggtgcatactggagaggagatgaaaggaagccaatgttgcaacggatatacggtactgcatgggagaatgaagagcagttgaaagcttaccttcacttcaaagaggaagccaagcgtcgagatcacaggcgcctagggcaagatcttgatttgttttctatacaggatgatgctggtgggggtctagttttctggcacccaaagggtgctatggtcagacacataattgaagatttctggaaaaaaattcacatgaaacgtggctatgatctactgtacacaccgcatgtggcaaaagctgatctttggaagatcagtggtcatttggatttctacaaagagaatatgtatgatcagatgagcgttgaggatgagctttatcagcttcggccaatgaattgtccttatcatattttggtgtacaaaagcaagcttcattcctatcgtgatttccctatcagggtagcagaattaggaactgtttatagatatgaactatctggaagtttacatggccttttccgtgttagaggtttcacacaggatgacgcacatatattttgtttagatgaccagattaaagatgagatcaggggtgtcctagatcttactgaagaaatactattgcaatttggttttgaaaagtatgaagtaaatttatctacaaggcctgaaaaggctgtgggagacgatgatatatgggaaaaagctacctctgctttagaagatgctttggatgataaaggttggacctatcaaattgatgatggaggaggtgccttttatgggccaaaggttgatatcaagattgaggatgctcttggtaggaagtggcaatgctcaaccatacaggttgatttcaatctaccacagcgttttgacatcacatatgttgactcaaatactgagaaaaggaggcctataatgatccatagagcagtgcttggatcattggaaagattctttggcattctcattgagcattatgctggtgattttccattatggctttctccaatacaagctcgagttttacctgttactgatgcccagcttgaatattgcaatgttgtgaccaacaaacttaaaatgtatggcatccgagctgaagtttgccatggtgagcgcctacctaaactaataagaaatgcagagaagcagaaaattccattaatggttgttgtgggctcaaaggaagttgaaacagaaacagtcactgttagatctaggtttggtggggagcttggaaccatgccagttgatgattttattgacagaataaagttgggaacagaaaacccaatatcattatga

SEQ ID NO: 9 - protein, 65 aa, Glycine max
  MISC_FEATURE (1)..(65): Gm_dTP - Dual targeting peptide comprising the first 65 amino acids of SEQ ID NO7
MATFAVATTLSTSSIAPLNARRGFGFGFGSFLSPTTKPKRPSITTTLAVATDSSVCFNPNPNPNP

SEQ ID NO: 10 - DNA, 195 bp, Glycine max
  misc_feature (1)..(195): DNA sequence encoding Gm_dTP (SEQ ID NO9)
atggctaccttcgcggttgcaacaacactttcgacatcttctatcgctccccttaatgcacgacgtggttttggttttggttttggctccttcctttctccaaccacaaaaccaaaacgcccctcaatcaccactaccctcgctgtcgccaccgactcttccgtttgtttcaatcccaaccccaaccctaaccct

SEQ ID NO: 11 - DNA, 235 bp, Artificial Sequence
  Synthetic - Cloning construct comprising the codon optimized DNA sequence of SEQ ID NO10
ttcgaattcggtctcaccatggctaccttcgcggttgcaacaacactttcgacatcttctatcgctccccttaatgcacgacgtggttttggttttggttttggctccttcctttctccaaccacaaaaccaaaacgcccctcaatcaccactaccctcgctgtcgccaccgactcttccgtttgtttcaatcccaaccccaaccctaaccctggaatgagagaccaagcttgaa

SEQ ID NO: 12 - DNA, 792 bp, Artificial Sequence
  Synthetic - Soybean codon optimized sequence of the aadA1 gene
atgagagaagctgttattgctgaagtttctactcaactttctgaagttgttggagttattgaaagacatcttgaacctactcttcttgctgttcatctttatggatctgctgttgatggaggacttaagccacattctgatattgatcttcttgttactgttactgttagacttgatgaaactactagaagagctcttattaatgatcttcttgaaacttctgcttctccaggagaatctgaaattcttagagctgttgaagttactattgttgttcatgatgatattattccatggagatatccagctaagagagaacttcaatttggagaatggcaaagaaatgatattcttgctggaatttttgaaccagctactattgatattgatcttgctattcttcttactaaggctagagaacattctgttgctcttgttggaccagctgctgaagaactttttgatccagttccagaacaagatctttttgaagctcttaatgaaactcttactctttggaattctccaccagattgggctggagatgaaagaaatgttgttcttactctttctagaatttggtattctgctgttactggaaagattgctccaaaggatgttgctgctgattgggctatggaaagacttccagctcaatatcaaccagttattcttgaagctagacaagcttatcttggacaagaagaagatagacttgcttctagagctgatcaacttgaagaatttgttcattatgttaagggagaaattactaaggttgttggaaagtga

SEQ ID NO: 13 - DNA, 829 bp, Artificial Sequence
  Synthetic - Cloning construct comprising the codon optimized sequence of SEQ ID NO12
ttcggtaccggtctcaaatgagagaagctgttattgctgaagtttctactcaactttctgaagttgttggagttattgaaagacatcttgaacctactcttcttgctgttcatctttatggatctgctgttgatggaggacttaagccacattctgatattgatcttcttgttactgttactgttagacttgatgaaactactagaagagctcttattaatgatcttcttgaaacttctgcttctccaggagaatctgaaattcttagagctgttgaagttactattgttgttcatgatgatattattccatggagatatccagctaagagagaacttcaatttggagaatggcaaagaaatgatattcttgctggaatttttgaaccagctactattgatattgatcttgctattcttcttactaaggctagagaacattctgttgctcttgttggaccagctgctgaagaactttttgatccagttccagaacaagatctttttgaagctcttaatgaaactcttactctttggaattctccaccagattgggctggagatgaaagaaatgttgttcttactctttctagaatttggtattctgctgttactggaaagattgctccaaaggatgttgctgctgattgggctatggaaagacttccagctcaatatcaaccagttattcttgaagctagacaagcttatcttggacaagaagaagatagacttgcttctagagctgatcaacttgaagaatttgttcattatgttaagggagaaattactaaggttgttggaaagtgagcttagagaccgcatgcctt

(19) United States
(12) Patent Application Publication
(10) Pub. No.: US 2022/0389440 A1
(43) Pub. Date: Dec. 8, 2022

(54) ALTERNATIVE TRANSIT PEPTIDES TO INCREASE PLANT TRANSFORMATION EFFICIENCY

(71) Applicant: WISCONSIN ALUMNI RESEARCH FOUNDATION, Madison, WI (US)

(72) Inventors: Michael Petersen, Merrimac, WI (US); Ray Collier, Fitchburg, WI (US); Edward Williams, Madison, WI (US)

(21) Appl. No.: 17/834,996

(22) Filed: Jun. 8, 2022

Related U.S. Application Data
(60) Provisional application No. 63/208,090, filed on Jun. 8, 2021.

(51) Int. Cl. C12N 15/82; C07K 14/415

(52) U.S. Cl. CPC C12N 15/8274; C07K 14/415; C07K 2319/60

(57) ABSTRACT

The present invention provides DNA constructs for producing transgenic plants. The DNA constructs encode a transit peptide that is targeted to the mitochondria and the chloroplasts within a cell. Also provided are plant cells and vectors comprising the DNA constructs. Methods of generating a transgenic plant using the vectors and constructs provided herein and methods of targeting a protein to mitochondria or chloroplasts in a cell.

### CROSS-REFERENCE TO RELATED APPLICATIONS

This application claims priority to U.S. Provisional Application No. 63/208,090 filed on Jun. 8, 2021, the contents of which are incorporated by reference in their entireties.

### SEQUENCE LISTING

A Sequence Listing accompanies this application and is submitted as an ASCII text file of the sequence listing named “960296_04292_ST25.txt” which is 23,916 bytes in size and was created on May 13, 2022. The sequence listing is electronically submitted via EFS-Web with the application and is incorporated herein by reference in its entirety.

### BACKGROUND

Transit peptides are commonly used in genetic engineering to deliver proteins to specific organelles within cells. For example, transit peptides are used to target selectable markers to specific organelles, such as the chloroplasts, to enable plant transformation and allow delivery of engineered proteins to the chloroplasts to allow these engineered proteins to avoid degradation in the cytoplasm of the cell and effect changes in plant photosynthesis, stress resistance, grain production and other changes to the metabolic functions of the plant. One commonly used selectable marker for dicot transformation is ANT(3″)(9), an adenylyltransferase that is encoded by the aadA1a gene, which confers resistance to streptomycin and spectinomycin. To achieve higher transformation rates, a chloroplast transit peptide (cTP) may be used in combination with this selectable marker. Alternative target peptides that produce even higher transformation rates or target peptides to different organelles are needed in the art.

### SUMMARY

In a first aspect, the present invention provides DNA constructs comprising a first expression cassette comprising a first heterologous promoter operably linked to a sequence encoding a transit peptide and a target protein. The transit peptide is at least 90% identical to SEQ ID NO:3 or SEQ ID NO:9.

In a second aspect, the present invention provides vectors comprising the DNA constructs described herein.

In a third aspect, the present invention provides plant cells comprising the DNA constructs described herein.

In a fourth aspect, the present invention provides methods of targeting a target protein to the chloroplasts and mitochondria of a plant cell. The methods comprise transforming the plant cell with a DNA construct described herein.

In a fifth aspect, the present invention provides methods of producing a transgenic plant. The methods comprise: (a) transforming an explant with a construct comprising a heterologous promoter operably linked to a sequence encoding a transit peptide described herein and a selectable marker; (b) contacting the explant with a selective agent; and (c) regenerating a transformed plant from the transgenic cells.

### BRIEF DESCRIPTION OF THE DRAWINGS

This patent or application contains at least one drawing executed in color. Copies of this patent or patent application publication with color drawing(s) will be provided by the Office upon request and payment of the necessary fee.

FIG. 1 shows a boxplot of transformation frequencies produced using alternative transit peptides and the optimized transformation protocol for soybean.

FIG. 2 shows tandem Tomato, targeted to the endoplasmic reticulum (tdTomato-ER), expression in T1 soybean seeds, demonstrating germline transmission. The seeds are visualized under LED spotlights. A roscolux 90 dark yellow-green LED is used to visualize tdTomato-ER. To reduce undesirable wavelengths of light, a colored filter is placed underneath the LED source. To visualize fluorescence, a second, excitation filter is affixed to the front of the cart. Here, Gamcolor245 is used to visualize tdTomato-ER. The top row shows seeds under ambient light (i.e., brightfield), the middle row shows seeds under green light with a red excitation filter (Gamcolor 245), and the bottom row shows seeds under a green light with a separate red excitation filter. From left to right, the seed samples were treated with DICOTBINARY19 (cTP aadA1a; labeled as WP345), DICOTBINARY20 (cytosolic aadA1a; labeled as WP346), DICOTBINARY22 (At_dTP aadA1a; labeled as WP348), and DICOTBINARY23 (Gm_dTP aadA1a; labeled as WP349). The photographs show T1 seeds segregating for tdTomato-ER (the null seeds are not glowing, while the positive seeds glow).

FIG. 3 shows PVC seed paddles containing 32 randomly sampled T1 seeds for segregation analysis of tdTomato-ER expression in T1 soybean seeds. The photograph on the left shows seeds under ambient light (i.e., brightfield), the photograph in the middle shows seeds under green light with a red excitation filter (Gamcolor 245), and the photograph on the right shows seeds under a green light with a separate red excitation filter.

FIG. 4 shows the results of transforming soybean using particle bombardment. (A) tdTomato-ER expression and segregation in T1 seedlings of soybean line WP1521-2a, which was generated using DICOTBOMB20 (At_dTP aadA1a). (B) GUS expression and segregation in T1 leaves of soybean lines generated using DICOTBOMB13 (cTP aadA1a; top) or DICOTBOMB20 (At_dTP aadA1a; bottom).

FIG. 5 shows the results of a pilot study using various amounts of imazapyr for selection at 5 weeks in W82 soybean value added explants (VAEs).

FIG. 6 shows the T0 plant phenotype of the first soybean VAE transformed with using At_dTP hra and imazapyr selection. (A) Photograph showing the T0 phenotype. (B) Image of a PCR gel showing a band of the expected size (87 bp) in lane 5, demonstrating that the T0 soybean plant expresses hra. (C) Images of leaf samples (left: negative control; right: T0 plant), demonstrating that the T0 plant expresses tdTomato-ER and GUS in its leaves. (D) Images of petiole sections (left: negative control; right: T0 plant), demonstrating that the T0 plant expresses tdTomato-ER and GUS in its vascular bundles, which indicates that the transfection likely penetrated into the L2/L3 layers of the meristem and that T1 positive seeds will likely be recovered. tdTomato-ER was visualized in the petiole sections and leaf samples using a Leica M165FC microscope and Leica Application Suite X (LAS X) software. For brightfield images, the iris was set to 64, and for tdTomato-ER images the iris was set to 100. For tdTomato-ER images, the lamp intensity was adjusted to the top setting, the exposure to approximately 500-2000 ms, and the gain to approximately 5. These settings appeared to be adequate to remove autoflorescence from the control samples and capture tdTomato-ER expression in transgenic samples.

FIG. 7 shows GUS expression and segregation in leaves of T1 progeny of the first soybean VAE transformed using At_dTP hra and imazapyr selection.

FIG. 8 shows tdTomato-ER expression and segregation in leaves of T1 progeny of the first soybean VAE transformed using At_dTP hra and imazapyr selection.

FIG. 9 shows soybean transformation using the optimized imazapyr selection protocol. (A) Photograph of soybean VAEs selected on 1 ppm imazapyr to 0.5 ppm imazapyr with elongated shoots (left), and on 2.5 ppm imazapyr to 0.5 ppm imazapyr without elongated shoots (right). (B) GUS expression and segregation in T1 leaves of soybean lines produced using the optimized imazapyr selection protocol.

FIG. 10 shows an imazapyr rooting assay for and segregating soybean after 2 weeks on rooting media with varying amounts of imazapyr.

FIG. 11 shows cowpea greening explants on spectinomycin media ˜4 weeks after inoculation with Ar18r12v/DICOTBINARY19 (cTP aadA1a; left) or Ar18r12v/DICOTBINARY22 (At_dTP aadA1a; right).

FIG. 12 shows tdTomato-ER expression in T0 cowpea plants produced using DICOTBINARY22 (At_dTP aadA1a). (A) Comparison of event WP1492-1a to a soybean negative control. (B) Comparison of the uniform tdTomato-ER expression in WP1492-1a (top) to the chimeric expression in WP1492-2a (bottom).

FIG. 13 shows tdTomato-ER and GUS expression in vascular bundles of petiole sections of T0 cowpea plants produced using DICOTBINARY22 (At_dTP aadA1a).

FIG. 14 demonstrates tdTomato-ER expression and segregation in T1 cowpea seeds. (A) Comparison of expression in various seeds produced using DICOTBINARY22 (At_dTP aadA1a). (B) Seeds from the WP1492-1a event.

FIG. 15 shows tdTomato-ER and GUS expression in Cannabis T0 events WP1331-2a and WP1331-1a, which were both transformed with DICOTBINARY22 (At_dTP aadA1a). (A) Images of whole plants, including a control cannabis plant (right) and T0 event WP1331-2a (left). The image on the left shows the plants under ambient light (brightfield), the image in the middle shows the plants under green light with a red excitation filter (Gamcolor 245), and the image on the right shows the plants under green light with a separate red excitation filter. (B) Images of petiole sections of WP1331-2a, demonstrating that the T0 plant expressed tdTomato-ER and GUS in its vascular bundles. These images were taken on a Leica M165FC microscope. (C) Image of a PCR gel in which aadA1a was amplified from 5 leaves of WP1331-1a. Three of the five leaves show the expected aadA1a amplicon (129 bp).

FIG. 16 shows tdTomato-ER expression in Cannabis lines produced using DICOTBINARY22 (At_dTP aadA1a). (A) tdTomato-ER expression and segregation in T1 seeds. (B) tdTomato-ER expression in T1 seedlings of Cannabis line WP1331-2a. A T1 positive seedling is shown on the left, and a T1 null is show on the right.

FIG. 17 shows tdTomato-ER expression in chickpeas. tdTomato-ER expression in T0 chickpea leaves (A) and roots (B) transformed with the constructs DICOTBINARY19 (cTP aadA1a), DICOTBINARY21 (ER aadA1a), and DICOTBINARY22 (At_dTP aadA1a).

FIG. 18 shows tdTomato-ER and GUS expression in roots and vascular bundles of petiole sections of chickpea T0 events WP1182-11a and -14a (DICOTBINARY19, cTP aadA1a) as compared to that of the L1 epidermal chimeric event WP1182-9a.

FIG. 19 shows tdTomato-ER expression in seed coats and cotyledons in T1 seed of germline chickpea events WP1182-11a and -14a and L1 epidermal chimeric event WP1182-9a (DICOTBINARY19, cTP aadA1a). The top row shows seeds under ambient light (i.e., brightfield), the middle row shows seeds under green light with a red excitation filter (Gamcolor 245), and the bottom row shows seeds under a green light with a separate red excitation filter. (A) Images of seed coats. From left to right: wild-type Sierra Chickpea, WP1182-9a, WP1182-11a, and WP1182-14a. Seed coats are maternally inherited T0 tissue, so even the L1 epidermal event (WP1182-9a) has glowing seed coats. (B) Images of cotyledons, from left to right: wild-type Sierra Chickpea, WP1182-9a, WP1182-11a, and WP1182-14a. Cotyledons are true T1 tissue, so L1 epidermal event WP1182-9a does not express tdTomato-ER in T1 cotyledons, but T1 cotyledons of germline events WP1182-11a and WP1182-14a express tdTomato-ER (as predicted by root/vascular bundle expression in FIG. 18).

FIG. 20 shows tdTomato-ER expression in T0 pinto bean plants transformed with the constructs DICOTBINARY19 (cTP aadA1a) and DICOTBINARY22 (At_dTP aadA1a).

FIG. 21 shows GUS expression in petiole sections of T0 pinto bean plants transformed with the constructs DICOTBINARY19 (cTP aadA1a) and DICOTBINARY22 (At_dTP aadA1a).

FIG. 22 shows tdTomato-ER expression in a T0 pea plant transformed with DICOTBINARY22 (At_dTP aadA1a).

FIG. 23 shows a schematic depicting the construct design described in Example 8, in which the plant selection marker gene is oriented such that its terminator is proximal to the T-DNA left border, and that transcription of the reporter gene is directed towards the T-DNA left border. In the schematic, each arrow represents a transcriptional unit comprising from 5′ to 3′: a promoter, a gene, and a terminator (including a polyadenylation signal). The arrowhead points in the direction of transcription. The T-DNA borders (RB=right border, LB=left border) delimit the T-DNA, with initiation occurring at the RB and termination occurring at the LB.

### DETAILED DESCRIPTION

The present invention provides DNA constructs for producing transgenic plants. The DNA constructs encode a transit peptide that is targeted to the mitochondria and the chloroplasts within a cell. Also provided are plant cells and vectors comprising the DNA constructs.

In the present application, the inventors tested the ability of two transit peptides to serve as alternatives to the proprietary chloroplast transit peptide (cTP). The first of these peptides is a “dual targeting peptide” (dTP) that targets both the mitochondria and the chloroplasts, and the second is an ER-targeting peptide/retention signal. DNA sequences encoding these transit peptides were cloned upstream of the aadA1a gene, which encodes an adenylyltransferase that confers resistance to streptomycin and spectinomycin, and these constructs were transformed into plants. The inventors tested two versions of the dual targeting peptide, one derived from Arabidopsis thaliana (At_dTP) and one derived from Glycine max (Gm_dTP). However, Gm_dTP failed to increase transformation frequency. Thus, only At_dTP was used in subsequent experiments.

While the inventors found that the ER-targeting peptide did not increase transformation frequencies in soybean plants, At_dTP produced greater transformation frequencies and recombinant protein expression than either of two control constructs, which contained either no transit peptide (i.e., the cytosolic construct) or a petunia-derived cTP. In fact, At_dTP produced transformation frequencies that were 1.4 to 3 times greater than those produced with cTP (see Example 1). Further, At_dTP enabled transformation of soybean with a particular resistance gene, highly resistant acetolactate synthase (hra), under conditions in which no transformed plants were recovered using the two control constructs (see Example 2). At_dTP also enabled the transformation of cowpea and chickpea, which were not successfully transformed using cTP under the same conditions (see Examples 3 and 5). Thus, the inventors have shown that this novel transit peptide is more effective at promoting transformation and allowing protein expression than the industry standard cTP.

The dual targeting peptide of the present invention, At_dTP, represents a promising means to increase plant transformation efficiencies and subsequent recombinant protein expression for genetic engineering applications. Importantly, this increase in transformation efficiency allows for more rapid regeneration of candidate transformed plant tissues, increased efficiency in identifying and growing transformed shoots and plants, reduced labor requirements, and reduced costs and ergonomic burden.

### DNA Constructs:

In a first aspect, the present invention provides DNA constructs comprising a first expression cassette comprising a first heterologous promoter operably linked to a sequence encoding a transit peptide and a target protein. As used herein, the term “DNA construct” refers a to recombinant polynucleotide, i.e., a polynucleotide that was formed by combining at least two polynucleotide components from different sources, natural or synthetic. For example, a construct may comprise the coding region of one gene operably linked to a promoter that is (1) associated with another gene found within the same genome, (2) from the genome of a different species, or (3) synthetic. Constructs can be generated using conventional recombinant DNA methods.

The DNA constructs of the present invention comprise an expression cassette. An “expression cassette” is a polynucleotide comprising a sequence encoding a polypeptide or a functional RNA as well as elements needed to express the encoded polypeptide or RNA (e.g., a promoter). The sequences controlling the expression of the gene are commonly referred to as a regulatory unit. Most parts of the regulatory unit are located upstream of coding sequence of the gene and are operably linked thereto. The expression cassette may also contain a downstream 3′ untranslated region comprising a polyadenylation site. The regulatory unit may be directly linked to the sequence to be expressed or separated therefrom by intervening DNA, e.g., by the 5′-untranslated region of a gene.

As used herein, the term “promoter” refers to a DNA sequence that regulates the expression of a polypeptide or functional RNA. Typically, a promoter is a regulatory region that is capable of binding RNA polymerase and initiating transcription of a downstream (3′ direction) sequence. However, a promoter may be located at the 5′ or 3′ end, within a coding region, or within an intron of a gene that it regulates. Promoters may be derived in their entirety from a native gene, may be composed of elements derived from multiple regulatory sequences found in nature, or may comprise synthetic DNA. A promoter is “operably linked” to a polynucleotide if the promoter is connected to the polynucleotide such that it can affect transcription of the polynucleotide. The promoters of the present invention are “heterologous”, meaning that they are not naturally associated with the host genome. It is understood by those skilled in the art that different promoters may direct the expression of a gene in different tissues or cell types, or at different stages of development, or in response to different environmental conditions. A number of promoters that are active in plant cells have been described in the literature. Such promoters include, but are not limited to, the nopaline synthase (NOS) and octopine synthase (OCS) promoters that are carried on T1 plasmids of Agrobacterium tumefaciens, caulimovirus promoters such as the cauliflower mosaic virus (CaMV) 19S and 35S promoters and the Figwort mosaic virus (FMV) 35S promoter, and the enhanced CaMV35S promoter (e35S). In some embodiments, the first heterologous promoter is a CaMV 35S promoter or a ubiquitin promoter.

The terms “transit peptide” and “targeting peptide” are used interchangeably herein to refer to a peptide that mediates post-translational translocation of a protein into a particular organelle (e.g., chloroplast, mitochondria, microbody, cyanelle). Transit peptides are commonly included in proteins as an N-terminal presequence. The transit peptides of the present invention are “dual targeting” peptides that direct translocation into both the mitochondria and the chloroplasts.

In the Examples, the inventors tested two versions of the dual targeting peptide (dTP): one derived from the Arabidopsis thaliana threonyl-tRNA synthetase (At2g04842.1; SEQ ID NO:1) and one derived from the Glycine max homolog of that protein (Glyma.19G191200.1; SEQ ID NO:7). The tested dTPs comprised the first 65 amino acids of these full-length peptides, i.e., SEQ ID NO:3 from Arabidopsis thaliana or SEQ ID NO:9 from Glycine max. Thus, the transit peptides used in the constructs of the present invention are at least 90% identical to SEQ ID NO:3 or SEQ ID NO:9. In some embodiments, the transit peptide is SEQ ID NO:3 or SEQ ID NO:9. Notably, the transit peptide of SEQ ID NO: 3 generated the highest transformation frequencies.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Protein and nucleic acid sequence identities are evaluated using the Basic Local Alignment Search Tool (“BLAST”), which is well known in the art (Karlin and Altschul, 1990, Proc. Natl. Acad. Sci. USA 87: 2267-2268; Altschul et al., 1997, Nucl. Acids Res. 25: 3389-3402). The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990), the disclosure of which is incorporated by reference in its entirety. The BLAST programs can be used with the default parameters or with modified parameters provided by the user.

The “target protein” used with the present invention can be any protein that one wishes to express in a plant. In some embodiments, the target protein confers a desirable trait, such as a trait of agronomic interest or a trait of industrial interest. Examples of traits of agronomic interest include, but are not limited to, disease, insect, or pest tolerance; herbicide tolerance; quality improvements, such as yield, fiber quality, nutritional enhancements (e.g., oil modification), or improved flavor; environmental or stress tolerances; any desirable changes in plant physiology, growth, development, morphology; and plant product production (e.g., starch, biopolymers, pharmaceutical peptides, etc.). Examples of traits of industrial interest include, but are not limited to, traits that are useful for carbon sequestration, biosensor development, or chemical/soil remediation.

In other embodiments, the target protein is a reporter protein or a selectable marker. A “reporter protein” is a protein that confers a characteristic that is easily identified or measured to an organism. A reporter protein is often used as an indication of whether a transgene has been taken up by or expressed in the cell or organism. Exemplary reporter proteins are known in the art and include, for example, β-glucuronidase (GUS), R-locus proteins, β-lactamase, luciferase, xy1E protein, α-amylase, tyrosinase, green fluorescence protein, other fluorescent proteins and α-galactosidase. A “selectable marker” is a protein that protects an organism from a selective agent that would normally kill it or prevent its growth. For example, in some embodiments, the selectable marker confers resistance to an antibiotic or an herbicide that would otherwise be toxic to the organism. In the Examples, the inventors demonstrate that the transit peptides described herein can be used to increase the efficiency of transforming a soybean plant with two genes that encode selectable markers: (1) aadA1a, which confers resistance to the antibiotic spectinomycin, and (2) hra, which confers resistance to the herbicide imazapyr. Thus, in some embodiments, the selectable marker is encoded by an aadA1a gene or a hra gene. In some embodiments, the selectable marker is encoded by SEQ ID NO:12, which is the soybean codon optimized aadA1a gene. In some embodiments, the selectable marker is encoded by SEQ ID NO:13, which is a cloning construct comprising SEQ ID NO:12.

In some embodiments, the DNA constructs further comprise a second expression cassette comprising a second heterologous promoter. In these embodiments, the DNA constructs may encode two target proteins: (1) a first target protein that is encoded by the first expression cassette and is expressed as a fusion protein with the transit peptide, and (2) a second target protein that is encoded by the second expression cassette such that it is expressed independently from the first target protein. For example, a construct may encode a first target protein that is a selectable marker (i.e., to allow for easy identification of cells that are successfully transformed with the construct) and a second target protein that confers a desirable trait that one wishes to transform into a plant. The second target protein may also be fused to a transit peptide if expression of the target requires trafficking to the chloroplast or mitochondria for effective expression. Alternatively, in these embodiments, the second heterologous promoter may be operably linked to a sequence that encodes an RNA molecule (e.g., antisense RNA, siRNA) that reduces the expression of an endogenous gene via gene silencing.

The constructs of the present invention are designed to express the transit peptide as a fusion protein with the target protein. A “fusion protein” is a protein consisting of at least two domains that are encoded by separate genes that have been joined such that they are transcribed and translated as a single unit, producing a single polypeptide. Thus, in some embodiments, the sequence encoding the transit peptide is translationally fused to the sequence encoding the target protein. The term “translationally fused” refers to two protein coding sequences that have been joined in a manner that retains the open reading frame of both protein coding sequences.

In some embodiments, the transit peptide is linked to the target protein by a linker peptide. As used herein, the term “linker peptide” refers to a peptide sequence that bridges two protein domains in a fusion protein. The linker may “flexible” such that it has no required fixed structure in solution and the adjacent protein segments are free to move relative to one another. The linker comprises 1 or more amino acid residues, preferably 1, 2, 3, 4, 5, 6, 7, 8, 9, 10, 11, 12, 13, 14, 15, 16, 17, 18, 19, or 20 or more residues. The linker may be an existing sequence provided by a protein domain included in the fusion protein or it may be provided by insertion of one or more amino acid residues between the protein domains of the fusion protein. The linker may comprise any amino acid sequence that does not substantially hinder the interaction of the polypeptides with their corresponding target molecules. The fusion proteins tested in the Examples comprise a simple two amino acid glycine-serine linker between the transit peptide and the target protein. Thus, in some embodiments, the linker comprises a glycine-serine linker.

The DNA construct used in the Examples comprises SEQ ID NO:5, which is a DNA sequence encoding the Arabidopsis thaliana dTP of SEQ ID NO:3 (At_dTP) that was codon-optimized for expression in soybean. Thus, in some embodiments, the DNA construct comprises SEQ ID NO:5. In some embodiments, the DNA construct comprises SEQ ID NO:6, which is a cloning construct comprising SEQ ID NO:5. In other embodiments, the DNA construct comprises SEQ ID NO:10, which is a DNA sequence encoding the Glycine max dTP (Gm_dTP) of SEQ ID NO:9. In some embodiments, the DNA construct comprises SEQ ID NO:11, which is a cloning construct comprising SEQ ID NO:10.

In a second aspect, the present invention provides vectors comprising the DNA constructs described herein. The term “vector” refers to a nucleic acid molecule capable of transporting another nucleic acid to which it is linked. Some vectors are capable of autonomous replication in a host cell into which they are introduced (e.g., bacterial vectors that include a bacterial origin of replication and episomal mammalian vectors). Other vectors can be integrated into the genome of a host cell such that they are replicated along with the host genome (e.g., viral vectors and transposons). Vectors suitable for use with the present invention comprise a DNA construct described herein and heterologous sequences necessary for proper propagation of the vector and expression of the encoded polypeptide. For example, the vector may contain genetic components that facilitate transformation of a plant cell or regulate the expression of the encoded proteins.

In some embodiments, the vector is a transfer DNA (T-DNA) vector for Agrobacterium-mediated transformation. A “T-DNA vector” is an artificial vector that is designed based on the naturally occurring T1 plasmid found in bacterial species of the genus Agrobacterium.

In some embodiments, the DNA constructs and vectors described herein are formulated for gene gun transformation. In gene gun transformation, a biolistic particle delivery system is used to deliver exogenous DNA into cells. Often, particles of a heavy metal (e.g., tungsten or gold) are coated with a DNA construct, and the particles are fired as micro-projectiles into cells using mechanical force. Thus, in some embodiments, the DNA constructs and vectors are coated onto heavy metal particles. For a description of gene gun transformation, see McCabe et al. (Bio/Technology (1988), 6:923-926), which is incorporated by reference in its entirety.

### Plants:

In a third aspect, the present invention provides plant cells comprising the constructs described herein. A “plant cell” is the basic structural unit of the plant, comprising a protoplast and a cell wall. A plant cell can be in the form of an isolated single cell or aggregate of cells (e.g., a friable callus or a cultured cell) or can be part of a higher organized unit. In the Examples, the inventors demonstrate that constructs encoding the transit peptide At_dTP can be used to transform soybean (see Examples 1 and 2), cowpea (see Example 3), hemp (see Example 4), chickpea (see Example 5), pinto bean (see Example 6), and pea (see Example 7). Thus, in some embodiments, the plant cell is a soybean, cowpea, hemp, chickpea, pinto bean, or pea plant cell. In some embodiments, the plant cells express the transit peptide and the target protein as a fusion protein. The transit peptides of the present invention are designed to target the fusion protein to the mitochondria and the chloroplasts. Thus, in some embodiments, the fusion protein is targeted to the mitochondria and the chloroplasts within the plant cell.

### Methods:

The transit peptides of the present invention are “dual targeting” peptides that simultaneously direct translocation of the target protein to both the mitochondria and the chloroplasts. Thus, in a fourth aspect, the present invention provides methods of targeting a target protein to the chloroplasts and mitochondria of a plant cell. The methods comprise transforming the plant cell with a DNA construct described herein.

The plant cell may be transformed using any method of plant transformation known in the art. Suitable methods for transforming nucleic acids into plant tissue include, without limitation, high velocity microprojection, microinjection, electroporation, direct DNA uptake, and bacterially mediated transformation. Bacteria known to mediate plant cell transformation include a number of species of Rhizobiaceae, including, but not limited to, Agrobacterium sp., Sinorhizobium sp., Mesorhizobium sp., and Bradyrhizobium sp. In some embodiments, the plant cells are transformed using Agrobacterium-mediated transformation. In this method, the DNA constructs are delivered into the plant cells using transferred (T)-DNA from bacterial cells through a bacterial type IV secretion system (T4SS).

Methods by which a plant is regenerated from tissue culture are known in the art and depend on the type of tissue that was used as an explant. See, e.g., Zambryski et al. (EMBO J. (1983) 2(12):2143-2150) for a description of such methods. See also U.S. Patent Publication No. US2019/0211347 and U.S. Patent Publication No. US2019/0208723, both of which are incorporated herein by reference in their entireties.

A variety of tissue culture media that support plant tissue growth and development when supplemented appropriately are known. These tissue culture media can be purchased commercially or can be prepared in-house. Those of skill in the art are aware that media and media supplements, such as nutrients and growth regulators, are usually optimized for the particular target crop or variety of interest. Tissue culture media may be supplemented with carbohydrates such as glucose, sucrose, maltose, mannose, fructose, lactose, galactose, dextrose, and combinations thereof. Suitable media include those utilized in the Examples, i.e., media based on Murashige and Skoog (MS) medium, Gamborg (B5) medium, and woody plant medium (WPM).

A “transgenic plant” is a plant into which one or more genes from another species have been introduced. A transgenic plant formed via Agrobacterium-mediated transformation typically contains a single recombinant DNA sequence inserted into one chromosome, which is referred to as a transgenic event. Such transgenic plants are hemizygous for the transgene. A transgenic plant that is homozygous for the transgene can be obtained by selfing an independent segregant transgenic plant that contains a single copy of the transgene (e.g., an T0 plant) to produce T1 seed. One fourth of the T1 seed produced will be homozygous with respect to the transgene. Germinating T1 seed produces plants that can be tested for zygosity, typically using a single-nucleotide polymorphism (SNP) assay or a thermal amplification assay that allows for the distinction between heterozygotes and homozygotes. To confirm the presence of the transgene in the transgenic plant, a variety of assays may be performed. Such assays include, for example, Southern blotting, northern blotting, western blotting, polymerase chain reaction (PCR)-based assays, Invader® assays, enzyme-linked immunoassays (ELISAs). Alternatively, if the transgene produces an observable phenotype, its presence may be confirmed by analyzing the phenotype of the whole regenerated plant.

Once a transgene has been introduced into a plant, that gene can be introduced into any plant that is sexually compatible with the first plant via crossing, i.e., without the need to directly transform the second plant. Therefore, the plants of the present invention include the progeny of any generation of a parent plant produced by the present methods, so long as the progeny comprise the transgene. Progeny can be produced by crossing a donor plant line that comprises the transgene with another plant line of interest. To accomplish this, one could, for example, perform the following steps: (a) plant seeds of a first (donor line) and second (line of interest) parent plant; (b) grow the seeds of the first and second parent plants into plants that bear flowers; (c) pollinate a flower from the first parent plant with pollen from the second parent plant; and (d) harvest seeds produced on the parent plant bearing the fertilized flower.

In the Examples, the inventors demonstrate that the use of a dual targeting peptide increases the efficiency of transformation in plants. Thus, in a fifth aspect, the present invention provides methods of producing a transgenic plant. The methods comprise: (a) transforming an explant with a construct comprising a heterologous promoter operably linked to a sequence encoding a transit peptide described herein and a selectable marker; (b) contacting the explant with a selective agent; and (c) regenerating a transformed plant from the transgenic cells.

The term “plant” is used herein to refer to a plant at any stage of development or to any part of a plant, including a plant cutting, a plant cell, a plant cell culture, a plant organ, a plant tissue, a plant seed, or a plantlet. Particularly useful parts of a plant include harvestable parts and parts that can be used for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like. The tissue culture will preferably be capable of regenerating plants.

Any species of plant may be used with the present invention, including monocot and dicot species. The term “monocot” is an abbreviation for monocotyledon and refers to plants that will have only one cotyledon, whereas the term “dicot” is an abbreviation for dicotyledon and refers to plants that will have two cotyledons. These two groups represent two divisions of the flowering plants. Suitable plants for use with the present invention include, for example, soybean, chickpea, wheat, barley, corn, cotton, pea, cannabis, cucurbits, tomato, potato, alfalfa, cowpea, dry beans, canola, and rice. In some embodiments, the plant is selected from a species that was transformed using the transit peptide At_dTP in the Examples, i.e., soybean, cowpea, cannabis, chickpea, pinto bean, and pea.

As used herein, the term “explant” refers to a cell, organ, or piece of tissue from a plant that has been transferred to a nutrient medium. Explants are the target material for transformation. They comprise meristematic tissue, which consists of undifferentiated cells that give rise to multiple plant structures (e.g., stems, roots, leaves, germline tissue, and seeds). Plant tissues that can be used as explants include, without limitation, embryos, cotyledons, hypocotyls, leaf bases, mesocotyls, plumules, protoplasts, and embryonic axes.

After the explants are transformed with the DNA construct, they are cultured in the presence of a selective agent. As used herein, the term “selective agent” refers to a compound that terminates or retards the growth of most of the plant cells into which the construct has not been delivered. Thus, a selective agent permits differentiation between cells that were successfully transformed with the construct and cells that were not transformed. Exemplary selective agents include, but are not limited to, kanamycin, hygromycin, streptomycin, chloramphenicol, ampicillin, erythromycin, spectinomycin, tetracycline, bialaphos, glyphosate, glufosinate, rifampicin, or dicamba, and the like. For example, in some embodiments, the selectable marker is encoded by the aadA1a gene and the selective agent is spectinomycin. In other embodiments, the selectable marker is encoded by the hra gene and the selective agent is imazapyr.

In some embodiments, the construct used in the methods further comprises a second heterologous promoter operably linked to a sequence that encodes a target protein, for example, a protein that confers a trait of agronomic interest. Alternatively, the construct may further comprise a second heterologous promoter operably linked to a sequence that encodes an RNA molecule (e.g., antisense RNA, siRNA) that reduces the expression of an endogenous gene via gene silencing.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1—Alternative Transit Peptides in Soybean (Glycine max, L.) Transformation with aadA1a/Spectinomycin Selection

We first attempted transformation of soybean meristem explants (of variety Williams82) that had been machine excised from imbibed seed and dried down to allow storage prior to transformation. For a description of soybean explants, see US Patent Publication No. US2019/0208723, which is incorporated by reference in its entirety. For this experiment, we used the following soybean transformation protocol:
Surface plate explants onto selective B5 media (without orientation) after co-culture
Overlay media at week 3 to delay Agrobacterium overgrowth and minimize transfers
Perform selection with 150 mg/L spectinomycin (corrected for activity) post co-culture
Transfer phenotype positive explants to 50 mg/L spectinomycin (active) at week 4-6 and then to non-selective B5 medium at week 8 for 2 weeks
Harvest shoots onto 150 mg/L spectinomycin rooting medium (BRM) at each stage if they are sufficiently elongated

In this pilot study, we compared the proprietary, petunia-derived chloroplast transit peptide (cTP) to two types of alternative transit peptides: “dual targeting peptides” (dTPs) that target both the mitochondria and the chloroplasts and an endoplasmic reticulum (ER)-targeting peptide. Two different dTPs were tested: one derived from the Arabidopsis thaliana threonyl-tRNA synthetase (At_dTP) and one derived from the Glycine max homolog of that protein (Gm_dTP). We also included a no transit peptide control that localizes to the cytosol in this experiment. DNA sequences encoding these transit peptides were cloned upstream of the aadA1a gene, which encodes an adenylyltransferase that confers resistance to streptomycin and spectinomycin, and were transformed into plants. We found that the construct comprising the Arabidopsis thaliana version of the dual targeting peptide (At_dTP) yielded the highest transformation rate, while the construct comprising the ER-targeting peptide yielded the lowest transformation rate (Table 1).

TABLE 1
Transformation metrics for the pilot study

| | | Subcellular | | | | | |
|---|---|---|---|---|---|---|---|
| | Promoter | location of | Visible | Regenerated | T0 | TF | TF |
| Binary | driving aadA1a | aadA1a | Explants | Explants | Plants | (vis) | (regen) |
| ST2 | GmUbi3 | cTP (chlor) | 530 | 364 | 10 | 1.9% | 2.8% |
| DB15 | 2X enhanced | cytosol | 526 | 361 | 10 | 1.9% | 2.8% |
| DB16 | CaMV35S + | endoplasmic | 533 | 366 | 2 | 0.4% | 0.5% |
| | TMV Ω | reticulum | | | | | |
| DB17 | | At_dTP (mito | 528 | 362 | 30 | 5.7% | 8.3% |
| | | & chlor) | | | | | |
| DB18 | | Gm_dTP | 404 | 276 | 12 | 3.0% | 4.3% |
| | | (mito & chlor) | | | | | |

ST = SOYTEST,
DB = DICOTBINARY

In this pilot study, the promoter used in the chloroplast transit peptide (cTP) construct was different from the promoter used in the other constructs, so we repeated the study using identical promoters in all the constructs and obtained similar results (Table 2). In this follow-up experiment, we used the same soybean transformation protocol that was used in the pilot test, but we embedded the explants (orienting apical meristems upward and radicals in media) directly after co-culture.

TABLE 2
Transformation metrics for the follow-up study

| | | Subcellular | | | | | |
|---|---|---|---|---|---|---|---|
| | Promoter driving | location of | Visible | Regenerated | T0 | TF % | TF % |
| Binary | aadA1a | aadA1a | Explants | Explants | Plants | (vis) | (regen) |
| DB19 | 2X enhanced | cTP (chlor) | 707 | 476 | 22 | 3.1 | 4.6 |
| DB20 | CaMV35S + | cytosol | 719 | 464 | 23 | 3.2 | 5.0 |
| DB21 | TMV Ω | endoplasmic | 541 | 380 | 0 | 0.0 | 0.0 |
| | | reticulum | | | | | |
| DB22 | | At_dTP (mito | 623 | 419 | 43 | 6.9 | 10.3 |
| | | & chlor) | | | | | |
| DB23 | | Gm_dTP | 685 | 455 | 19 | 2.8 | 4.2 |
| | | (mito & chlor) | | | | | |

DB = DICOTBINARY

We ran further tests to optimize the soybean value added explant (VAE) transformation protocol using the cTP construct (DICOTBINARY19). We found that embedding the explants in B5 media with their apical meristems oriented upward directly after co-culture (as opposed to surface plating without orientation) produced a large increase in transformation frequency. Based on this result, we eliminated the liquid overlay steps and reduced the number of regeneration transfers to three, producing the following optimized soybean transformation protocol:
Co-culture for 4-5 days in 2.5 ml INO medium with 1 ppm thidiazuron (TDZ)/plantcon
Embed explants (oriented apical meristems upward and radicals in media) directly after co-culture and for subsequent transfers for greening, phenotype positive explants on spectinomycin
Perform selection on 150 mg/L spectinomycin (corrected for activity) post co-culture for 3-4 weeks
Transfer phenotype positive explants to 50 mg/L spectinomycin (active) for 3-4 weeks and then to non-selective B5 medium for 2 weeks
Harvest shoots onto 150 mg/L spectinomycin in dicot basal rooting medium at each stage if sufficiently elongated
   The media used in this optimized protocol are detailed in Tables 3-5, below.

TABLE 3
Dicot INO medium composition

| Ingredients and notes | Amount to add per liter (grams) |
|---|---|
| Phytotechnology Laboratories | 1.284 |
| B5 salts G398 | |
| Glucose | 30 |
| MES hydrate (Alfa Aesar CAS | 2.8 |
| 4432-31-9) | |
| pH to 5.4 with 1N KOH | |
| Autoclave | |

TABLE 4
Gamborg True B5 medium composition

| Ingredients and notes | Amount to add per liter (grams) |
|---|---|
| Phytotechnology Laboratories | 3.21 |
| B5 salts G398 | |
| Sucrose | 20 |
| Cleary's 3336 (50WP) | 0.06 |
| Ca Gluconate | 1.29 |
| pH to 5.8 with 1N KOH | |
| Phytagel | 3.50 |
| Autoclave | |
| Add the following fresh before | |
| use: | |
| Timetin (150 mg/ml stock) | Use 1 mL per Liter (150 mg/L) |
| Cefotaximine (100 mg/ml | Use at 2 ml per Liter (200 mg/L) |
| stock) | |
| Carbenicillin (100 mg/ml stock) | Use at 4 ml per Liter (400 mg/L) |
| Selective agent | as needed |

TABLE 5
Dicot basal rooting medium (BRM) composition

| Ingredients and notes | Amount to add per liter (grams) |
|---|---|
| MS Salts no vitamins M524 | 2.15 |
| Myo-inositol | 0.1 |
| Sucrose | 30 |
| pH 5.8 with KOH | |
| Agar, Sigma A7921 | 8 |
| Autoclave | |
| Add after autoclaving: | |
| Cysteine (100 mg/ml) - make | Use at 1.0 ml per Liter (100 mg/L) |
| stock fresh | |
| Cefotaxime (100 mg/ml) | Use at 2.0 ml per Liter (200 mg/L) |
| IAA (1 mg/ml) | Use at 0.1 ml per Liter (0.1 mg/L) |
| MS Vitamins (1000X) | Use at 1.0 ml per Liter |
| Selection | as needed |

We tested the optimized protocol against the original surface plating protocol with a construct encoding cTP-aadA1a and found that it produced higher transformation frequencies. Specifically, the surface plating method produced a transformation frequency of 0.1%, while the optimized protocol produced a transformation frequency of 3-5% (Table 6). The soybean plants used in this experiment were single seed descent of the Williams82 and 3613N varieties.

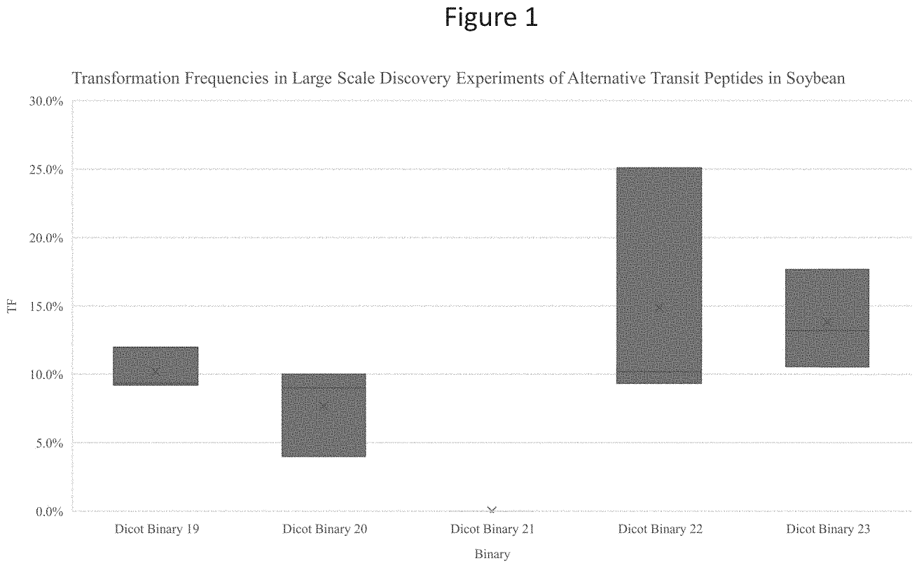

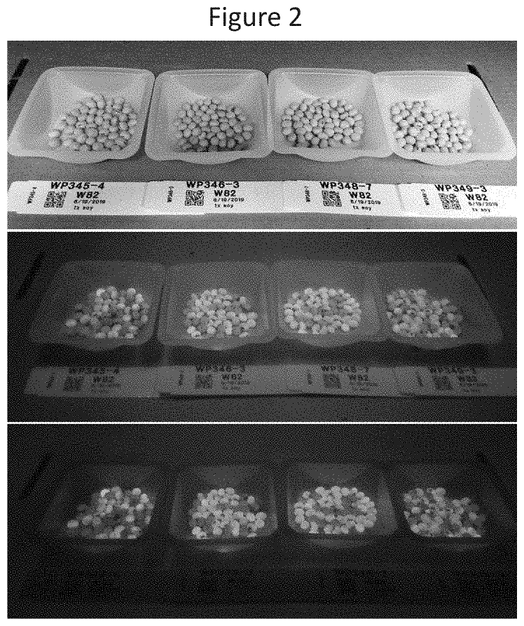

Using the optimized soybean transformation protocol, we tested the alternative transit peptides in a larger experiment with three replicates and produced similar results as in the initial tests (Table 7). As is shown in FIG. 1, the DICOTBINARY22 construct comprising the bi-organellar transit peptide (At_dTP) yielded the highest transformation rate (TF). The soybean plants used in this experiment were single seed descent of the Williams82 variety.

In Tables 6 and 7, the number of visible explants is based on a sample taken of the VAE batch after excision. A mass is measured, and a technician counts the number of explants under a microscope, giving the batch a visible explant metric of “visible explants/gram.” This metric is used to estimate how many visible explants are used in a given experiment. However, not all visible explants have intact meristematic tissue due to mechanical damage. Thus, we also count the subset of “regenerable explants” after transferring the explants off co-culture. The term “greening explants” refers to explants that stay green through spectinomycin selection, which bleaches non-transgenic tissues. Thus, greening explants, which are counted at the different transfer steps, are an early indicator of “phenotype positive” explants. We also harvest shoots and obtain rooted shoots (i.e., T0 plants) at each transfer step. An elongated greening shoot is a good sign that a plant is transgenic, and a shoot that roots in the presence of selection suggests that the event is germline (i.e., will give rise to T1 positive seed). “TF(vis)” is the transformation frequency based on visible explants, which is calculated by dividing the total number of T0 events by the number of initial visible explants. “TF(regen)” is the transformation frequency based on regenerable explants, which is calculated by dividing the total number of T0 events by the number of initial regenerable explants. The abbreviation “DB” stands for dicot binary.

TABLE 6
Transformation metrics for the optimized soybean transformation protocol

| | | | | | | T0 plants | |
|---|---|---|---|---|---|---|---|
| | | | | Regenerable | | from 150 | # Greening |
| | | | Visible explants | explants to | Shoots from | ppm spec | explants at wk |
| | | | to 150 ppm | 150 ppm | 150 ppm | (rooted on | 4 transferred to |
| Cultivar | Treatment | Binary | spec B5 | spec B5 | spec B5 | 150 ppm spec) | 50 ppm spec B5 |
| SSW82 | explants + | DB19 | 65 | 50 | 0 | 0 | 4 |
| | debris surface | | | | | | |
| | plated | | | | | | |
| SSW82 | pure singulated | DB19 | 100 | 100 | 2 | 2 | 26 |
| | explants | | | | | | |
| SSW82 | singulated | DB19 | 126 | 93 | 8 | 8 | 32 |
| | explants | | | | | | |
| 3613N | singulated | 36222 | 600 | 450 | 2 | 2 | 83 |
| | explants | | | | | | |

| | | | # Greening | | | | |
|---|---|---|---|---|---|---|---|
| | Shoots from | T0 plants from 50 | explants at wk 8 | Shoots from | T0 plants from non- | | |
| | 50 ppm | ppm spec (rooted | transferred to | non-selective | selective B5 (rooted | TF | TF |
| Cultivar | spec B5 | on 150 ppm spec) | non-selective B5 | B5 | on 150 ppm spec) | (vis) | (regen) |
| SSW82 | 0 | 0 | 1 | 1 | 1 | 1.5% | 2.0% |
| SSW82 | 5 | 5 | 5 | 5 | 5 | 12.0% | 12.0% |
| SSW82 | 5 | 5 | 17 | 4 | 4 | 13.5% | 18.3% |
| 3613N | 11 | 11 | 55 | 8 | 7 | 3.3% | 4.4% |

TABLE 7
Transformation metrics for the larger scale experiment with the optimized soybean transformation protocol. Data for individual replicates
are shown in the top portion of the table, and data for the combined replicates are shown in the bottom portion of the table.

| | | | | | | | | | Total T0 | | | |
|---|---|---|---|---|---|---|---|---|---|---|---|---|
| | | | T0 plants | | T0 plants | | T0 plants | | plants | | | |
| | | Shoots | (rooting | Shoots | (rooting | Shoots | (rooting | | (rooting | | | |
| | Ex- | from 150 | on 150 | from 50 | on 150 | from | on 150 | Total | on 150 | Shooting | Rooting | |
| Binary | plants | ppm spec | spec BRM) | ppm spec | spec BRM) | B5 | spec BRM) | Shoots | spec BRM) | % | % | TF |
| DB19 | 425 | | | 36 | 33 | 23 | 18 | 59 | 51 | 13.9% | 86.4% | 12.0% |
| DB20 | 300 | | | 22 | 6 | 10 | 6 | 32 | 12 | 10.7% | 37.5% | 4.0% |
| DB21 | 300 | | | 0 | 0 | 0 | 0 | 0 | 0 | 0.0% | n/a | 0.0% |
| DB22 | 275 | | | 46 | 45 | 28 | 24 | 74 | 69 | 26.9% | 93.2% | 25.1% |
| DB23 | 300 | | | 49 | 40 | 24 | 13 | 73 | 53 | 24.3% | 72.6% | 17.7% |
| DB19 | 300 | 12 | 10 | 9 | 9 | 9 | 9 | 30 | 28 | 10.0% | 93.3% | 9.3% |
| DB20 | 300 | 18 | 11 | 22 | 11 | 8 | 5 | 48 | 27 | 16.0% | 56.3% | 9.0% |
| DB21 | 250 | 0 | 0 | 0 | 0 | 0 | 0 | 0 | 0 | 0.0% | n/a | 0.0% |
| DB22 | 275 | 4 | 4 | 18 | 17 | 7 | 7 | 29 | 28 | 10.5% | 96.6% | 10.2% |
| DB23 | 275 | 11 | 11 | 15 | 14 | 7 | 4 | 33 | 29 | 12.0% | 87.9% | 10.5% |
| DB19 | 250 | | | 13 | 11 | 15 | 12 | 28 | 23 | 11.2% | 82.1% | 9.2% |
| DB20 | 250 | | | 25 | 10 | 28 | 15 | 53 | 25 | 21.2% | 47.2% | 10.0% |
| DB21 | 250 | | | 0 | 0 | 0 | 0 | 0 | 0 | 0.0% | n/a | 0.0% |
| DB22 | 225 | | | 12 | 10 | 15 | 11 | 27 | 21 | 12.0% | 77.8% | 9.3% |
| DB23 | 250 | | | 29 | 23 | 16 | 10 | 45 | 33 | 18.0% | 73.3% | 13.2% |
| DB19 | 975 | 12 | 10 | 58 | 53 | 47 | 39 | 117 | 102 | 12.0% | 87.2% | 10.5% |
| DB20 | 850 | 18 | 11 | 69 | 27 | 46 | 26 | 133 | 64 | 15.6% | 48.1% | 7.5% |
| DB21 | 800 | 0 | 0 | 0 | 0 | 0 | 0 | 0 | 0 | 0.0% | n/a | 0.0% |
| DB22 | 775 | 4 | 4 | 76 | 72 | 50 | 42 | 130 | 118 | 16.8% | 90.8% | 15.2% |
| DB23 | 825 | 11 | 11 | 93 | 77 | 47 | 27 | 151 | 115 | 18.3% | 76.2% | 13.9% |

In addition to the aadA1a reporter gene, most of our test constructs (e.g., DICOTBINARY22) contain transcription units encoding a non-lethal scorable marker (i.e., tdTomato-ER) and a lethal scorable marker (i.e., GUS). Inclusion of these additional markers allows corroborative data to be collected during the course of the experiment. For example, the fluorescent signal of tdTomato-ER (which is a tandem Tomato marker that is localized to the endoplasmic reticulum for enhanced brightness) can be detected without harming the plants prior their use in the lethal GUS reporter assay. Thus, we next confirmed germline transmission in the plants generated in the second, follow-up experiment (Table 2) using non-destructive whole seed imaging of tdTomato-ER in T1 seed (FIG. 2, Table 8).

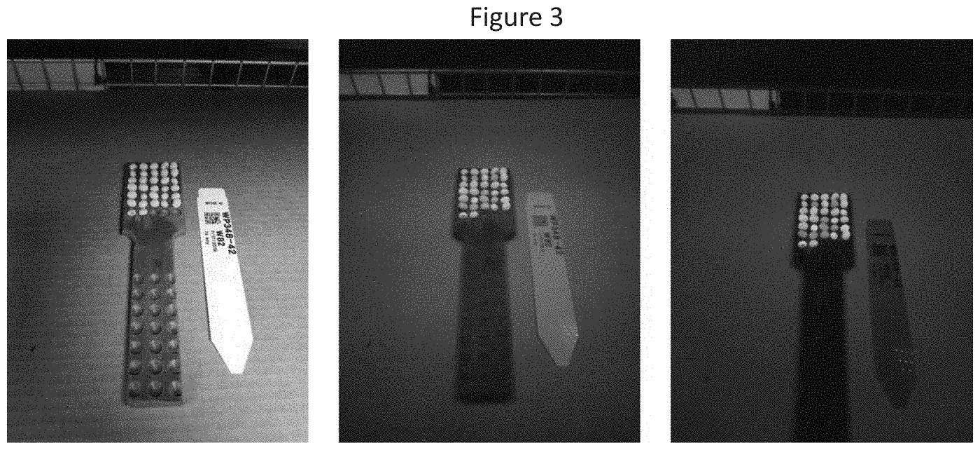

We then assessed segregation by randomly sampling about 32 T1 seeds per line using a PVC block bored with indentions to accommodate a soybean seed and imaging these singulated seeds for tdTomato-ER expression (FIG. 3). tdTomato-ER-positive seeds were counted and ratio compared. A 3:1 ratio was expected based on Mendelian segregation of a single copy transgene in the parental hemizygotic event (Table 9). Germline transmission was high (greater than 80%) with all of the alternative transit peptides, but the cTP and At_dTP peptides had the highest percentage of lines possibly segregating 3:1 by chi-square.

TABLE 8
Summary of germline transmission and segregation of tdTomato-ER in T1 seed from the follow-up experiment

| | | | | | | | | | % Lines | % Lines |
|---|---|---|---|---|---|---|---|---|---|---|
| | | | | | | | | # Lines | with | possible |
| | | | | | | # Lines | # Lines | possible | tdTom+ | segregating |
| | | | Subcellular | | | examined | with | segregating | T1 seed | 3:1 |
| | | | location of | T0 | | for T1 | tdTom+ | 3:1 by chi- | (% | by chi- |
| Species | Genotype | Binary | aadA1a | plants | Workplan | tdTom | T1 seed | square | germline) | square |
| Soybean | W82 | DB19 | cTP | 22 | WP345 | 16 | 14 | 7 | 88% | 44% |
| Soybean | W82 | DB20 | cytosol | 23 | WP346 | 18 | 15 | 4 | 83% | 22% |
| Soybean | W82 | DB22 | At_dTP | 43 | WP348 | 35 | 32 | 14 | 91% | 40% |
| Soybean | W82 | DB23 | Gm_dTP | 19 | WP349 | 16 | 14 | 3 | 88% | 19% |

TABLE 9
Raw data used to generate Table 8.

| | Subcellular | | | | | | |
|---|---|---|---|---|---|---|---|
| | location of | | | | Expected RFP | Expected RFP | |
| Line | aadA1a | # T1 seed | # RFP pos | # RFP neg | pos (3:1) | neg (3:1) | Chi square p |
| WP345-3 | cTP | 32 | 11 | 21 | 24 | 8 | 0.0000 |
| WP345-4 | cTP | 32 | 20 | 12 | 24 | 8 | 0.1025 |
| WP345-5 | cTP | 32 | 0 | 32 | 24 | 8 | 0.0000 |
| WP345-6 | cTP | 32 | 11 | 21 | 24 | 8 | 0.0000 |
| WP345-7 | cTP | 32 | 22 | 10 | 24 | 8 | 0.4142 |
| WP345-8 | cTP | 32 | 20 | 12 | 24 | 8 | 0.1025 |
| WP345-9 | cTP | 32 | 0 | 32 | 24 | 8 | 0.0000 |
| WP345-10 | cTP | 32 | 8 | 24 | 24 | 8 | 0.0000 |
| WP345-11 | cTP | 32 | 27 | 5 | 24 | 8 | 0.2207 |
| WP345-12 | cTP | 32 | 13 | 19 | 24 | 8 | 0.0000 |
| WP345-14 | cTP | 32 | 13 | 19 | 24 | 8 | 0.0000 |
| WP345-15 | cTP | 32 | 12 | 20 | 24 | 8 | 0.0000 |
| WP345-16 | cTP | 32 | 20 | 12 | 24 | 8 | 0.1025 |
| WP345-17 | cTP | 32 | 25 | 7 | 24 | 8 | 0.6831 |
| WP345-18 | cTP | 32 | 16 | 16 | 24 | 8 | 0.0011 |
| WP345-19 | cTP | 32 | 27 | 5 | 24 | 8 | 0.2207 |
| WP346-3 | cytosol | 32 | 22 | 10 | 24 | 8 | 0.4142 |
| WP346-5 | cytosol | 32 | 2 | 30 | 24 | 8 | 0.0000 |
| WP346-6 | cytosol | 32 | 25 | 7 | 24 | 8 | 0.6831 |
| WP346-7 | cytosol | 32 | 4 | 28 | 24 | 8 | 0.0000 |
| WP346-8 | cytosol | 32 | 0 | 32 | 24 | 8 | 0.0000 |
| WP346-10 | cytosol | 32 | 26 | 6 | 24 | 8 | 0.4142 |
| WP346-11 | cytosol | 32 | 2 | 30 | 24 | 8 | 0.0000 |
| WP346-12 | cytosol | 32 | 13 | 19 | 24 | 8 | 0.0000 |
| WP346-13 | cytosol | 32 | 16 | 16 | 24 | 8 | 0.0011 |
| WP346-14 | cytosol | 32 | 0 | 32 | 24 | 8 | 0.0000 |
| WP346-15 | cytosol | 32 | 19 | 13 | 24 | 8 | 0.0412 |
| WP346-16 | cytosol | 32 | 16 | 16 | 24 | 8 | 0.0011 |
| WP346-17 | cytosol | 32 | 15 | 17 | 24 | 8 | 0.0002 |
| WP346-18 | cytosol | 32 | 15 | 17 | 24 | 8 | 0.0002 |
| WP346-19 | cytosol | 32 | 6 | 26 | 24 | 8 | 0.0000 |
| WP346-21 | cytosol | 32 | 17 | 15 | 24 | 8 | 0.0043 |
| WP346-22 | cytosol | 32 | 0 | 32 | 24 | 8 | 0.0000 |
| WP346-23 | cytosol | 16 | 11 | 5 | 12 | 4 | 0.5637 |
| WP348-6 | At_dTP | 32 | 5 | 27 | 24 | 8 | 0.0000 |
| WP348-7 | At_dTP | 32 | 23 | 9 | 24 | 8 | 0.6831 |
| WP348-8 | At_dTP | 32 | 16 | 16 | 24 | 8 | 0.0011 |
| WP348-10 | At_dTP | 32 | 17 | 15 | 24 | 8 | 0.0043 |
| WP348-11 | At_dTP | 32 | 16 | 16 | 24 | 8 | 0.0011 |
| WP348-12 | At_dTP | 32 | 18 | 14 | 24 | 8 | 0.0143 |
| WP348-13 | At_dTP | 32 | 10 | 22 | 24 | 8 | 0.0000 |
| WP348-15 | At_dTP | 32 | 5 | 27 | 24 | 8 | 0.0000 |
| WP348-16 | At_dTP | 32 | 5 | 27 | 24 | 8 | 0.0000 |
| WP348-17 | At_dTP | 32 | 20 | 12 | 24 | 8 | 0.1025 |
| WP348-18 | At_dTP | 32 | 21 | 11 | 24 | 8 | 0.2207 |
| WP348-19 | At_dTP | 32 | 22 | 10 | 24 | 8 | 0.4142 |
| WP348-20 | At_dTP | 32 | 0 | 32 | 24 | 8 | 0.0000 |
| WP348-21 | At_dTP | 32 | 27 | 5 | 24 | 8 | 0.2207 |
| WP348-22 | At_dTP | 32 | 17 | 15 | 24 | 8 | 0.0043 |
| WP348-23 | At_dTP | 32 | 24 | 8 | 24 | 8 | 1.0000 |
| WP348-24 | At_dTP | 32 | 21 | 11 | 24 | 8 | 0.2207 |
| WP348-25 | At_dTP | 32 | 17 | 15 | 24 | 8 | 0.0043 |
| WP348-26 | At_dTP | 32 | 18 | 14 | 24 | 8 | 0.0143 |
| WP348-27 | At_dTP | 32 | 0 | 32 | 24 | 8 | 0.0000 |
| WP348-28 | At_dTP | 32 | 18 | 14 | 24 | 8 | 0.0143 |
| WP348-29 | At_dTP | 32 | 12 | 20 | 24 | 8 | 0.0000 |
| WP348-30 | At_dTP | 32 | 23 | 9 | 24 | 8 | 0.6831 |
| WP348-31 | At_dTP | 32 | 23 | 9 | 24 | 8 | 0.6831 |
| WP348-32 | At_dTP | 32 | 22 | 10 | 24 | 8 | 0.4142 |
| WP348-33 | At_dTP | 32 | 28 | 4 | 24 | 8 | 0.1025 |
| WP348-34 | At_dTP | 32 | 12 | 20 | 24 | 8 | 0.0000 |
| WP348-36 | At_dTP | 32 | 29 | 3 | 24 | 8 | 0.0412 |
| WP348-37 | At_dTP | 32 | 25 | 7 | 24 | 8 | 0.6831 |
| WP348-38 | At_dTP | 32 | 18 | 14 | 24 | 8 | 0.0143 |
| WP348-39 | At_dTP | 32 | 19 | 13 | 24 | 8 | 0.0412 |
| WP348-40 | At_dTP | 32 | 0 | 32 | 24 | 8 | 0.0000 |
| WP348-41 | At_dTP | 32 | 14 | 18 | 24 | 8 | 0.0000 |
| WP348-42 | At_dTP | 32 | 24 | 8 | 24 | 8 | 1.0000 |
| WP348-43 | At_dTP | 32 | 24 | 8 | 24 | 8 | 1.0000 |
| WP349-2 | Gm_dTP | 32 | 12 | 20 | 24 | 8 | 0.0000 |
| WP349-3 | Gm_dTP | 32 | 19 | 13 | 24 | 8 | 0.0412 |
| WP349-4 | Gm_dTP | 32 | 17 | 15 | 24 | 8 | 0.0043 |
| WP349-5 | Gm_dTP | 32 | 12 | 20 | 24 | 8 | 0.0000 |
| WP349-6 | Gm_dTP | 32 | 0 | 32 | 24 | 8 | 0.0000 |
| WP349-7 | Gm_dTP | 32 | 27 | 5 | 24 | 8 | 0.2207 |
| WP349-9 | Gm_dTP | 32 | 5 | 27 | 24 | 8 | 0.0000 |
| WP349-10 | Gm_dTP | 32 | 13 | 19 | 24 | 8 | 0.0000 |
| WP349-11 | Gm_dTP | 32 | 0 | 32 | 24 | 8 | 0.0000 |
| WP349-12 | Gm_dTP | 32 | 24 | 8 | 24 | 8 | 1.0000 |
| WP349-14 | Gm_dTP | 32 | 13 | 19 | 24 | 8 | 0.0000 |
| WP349-15 | Gm_dTP | 32 | 8 | 24 | 24 | 8 | 0.0000 |
| WP349-16 | Gm_dTP | 32 | 15 | 17 | 24 | 8 | 0.0002 |
| WP349-17 | Gm_dTP | 32 | 28 | 4 | 24 | 8 | 0.1025 |
| WP349-18 | Gm_dTP | 32 | 14 | 18 | 24 | 8 | 0.0000 |
| WP349-19 | Gm_dTP | 32 | 19 | 13 | 24 | 8 | 0.0412 |

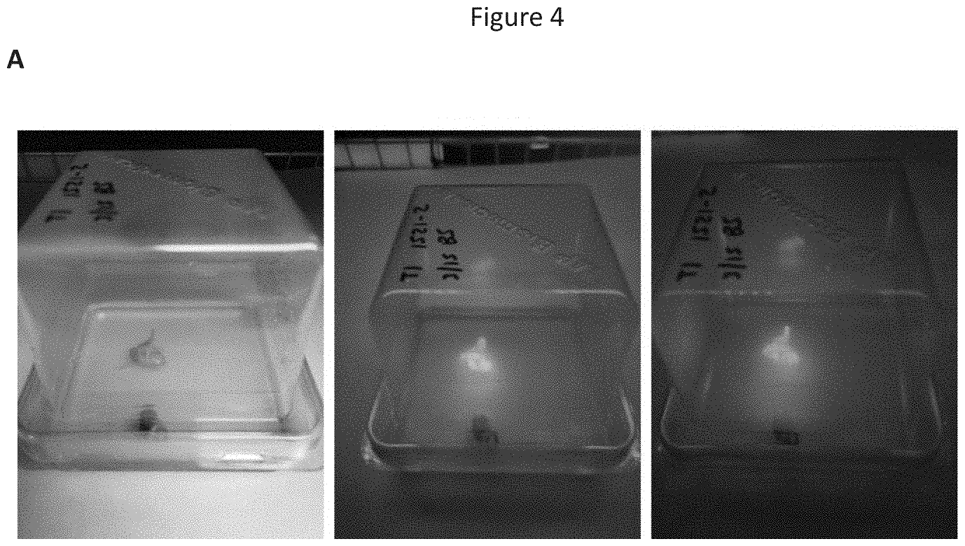

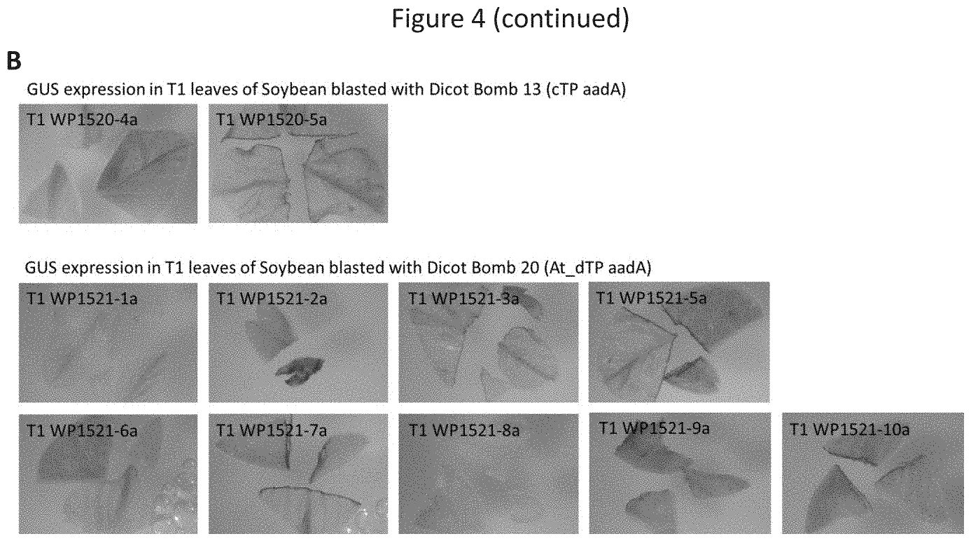

We also tested the At_dTP transit peptide in particle-mediated transformation of soybean VAEs (single seed descent variety of Williams82) using the PDS-1000 Helium gun. In this experiment, we used the constructs DICOTBOMB13 (cTP aadA1a) and DICOTBOMB20 (At_dTP aadA1a). A 6 cm distance from the microcarrier (which contained a 5 μl drop of the DNA-gold prep) to the soybean explants was used. The T1 metrics (germline frequencies and segregation data) for this experiment are presented in Tables 10-13 and in FIG. 4. These data demonstrate germline transmission of GUS in soybean using At_dTP aadA with particle bombardment.

TABLE 10
Transformation metrics for particle-mediated transformation with DICOTBOMB20 (At_dTP aadA1a). The bead prep metric gives
the amount of DNA precipitated on a given mass of gold. 1350 psi refers to the rupture disk in the PDS-1000 Helium gun.

| | | | | | | # Greening to | | | | |
|---|---|---|---|---|---|---|---|---|---|---|
| | | | | | # Explants | 50 ppm spec | | | | |
| | | | | | to 150 mg/L | B5 (active) | | # | | |
| | | | Blasting | | spec B5 | 3 wks post- | # | Rooted | | % Germ- |
| Experiment | Preculture | Bead prep | conditions | Rest | (active) | blast | Shoots | shoots | TF | line |
| Soybean | EJW1 with 8 g/L | 1.2 ng | 1X blast; 1 cm | EJW1 with 8 g/L | 95 | 18 | 7 | 3 | 3.2% | 67% |
| VAE 3/17-3 | agarose I; 28 C. | DNA/ug | gap; 1350 psi; | agarose I; 28 C. | | | | | | |
| | 16/8 photoperiod | 0.6 um | 6 cm distance | 16/8 photoperiod | | | | | | |
| | | gold | | | | | | | | |
| | | BOMB | | | | | | | | |
| | | 20 | | | | | | | | |
| Soybean | EJW1 with 8 g/L | 1.2 ng | 1X blast; 1 cm | EJW1 with 8 g/L | 13 | 1 | 1 | 1 | 7.7% | 100% |
| VAE 3/17- | agarose I; 28 C. | DNA/ug | gap; 1100 psi; | agarose I; 28 C. | | | | | | |
| 3B | 16/8 photoperiod | 0.6 um | 6 cm distance | 16/8 photoperiod | | | | | | |
| | | gold | | | | | | | | |
| | | BOMB | | | | | | | | |
| | | 20 | | | | | | | | |
| Soybean | INO TDZ with | 1.2 ng | 1X blast; 1 cm | INO TDZ with | 71 | 5 | 3 | 3 | 4.2% | 100% |
| VAE 3/17-4 | 8 g/L agarose I; | DNA/ug | gap; 1350 psi; | 8 g/L agarose I; | | | | | | |
| | 28 C. 16/8 | 0.6 um | 6 cm distance | 28 C. 16/8 | | | | | | |
| | photoperiod | gold | | photoperiod | | | | | | |
| | | BOMB | | | | | | | | |
| | | 20 | | | | | | | | |
| Soybean | Mod TDZ OR | 1.2 ng | 1X blast; 1 cm | Mod TDZ OR | 27 | 4 | 0 | 0 | 0.0% | n/a |
| VAE 3/17-5 | v33; 28 C. 16/8 | DNA/ug | gap; 1100 psi; | v33; 28 C. 16/8 | | | | | | |
| | photoperiod | 0.6 um | 6 cm distance | photoperiod | | | | | | |
| | | gold | | | | | | | | |
| | | BOMB | | | | | | | | |
| | | 20 | | | | | | | | |

TABLE 11
Transformation metrics for particle-mediated transformation with DICOTBOMB20 (At_dTP aadA1a) or DICOTBOMB13 (cTP aadA1a).

| | | | | Initial Explants | | | | |
|---|---|---|---|---|---|---|---|---|
| | | | | on 150 ppm spec | Shoots | T0 | | |
| Experiment | Preculture conditions | Construct | Rest conditions | B5 | harvested | plants | TF | % Germline |
| Soybean VAE | VAE, precultured on EJW1 | Dicot | EJW1 o/n at 28 C. 16/8 | 48 | 2 | 1 | 2.1% | n/a (no T1 |
| 2/25-1 | o/n at 15 C. dark | Bomb 13 | photoperiod | | | | | seed set) |
| Soybean VAE | VAE precultured on EJW1 | Dicot | EJW1 with 8 g/L agarose I | 43 | 2 | 2 | 4.7% | 50% |
| 2/25-2 | with 8 g/L agarose I o/n at | Bomb 13 | o/n at 28 C. 16/8 | | | | | |
| | 15 C. dark | | photoperiod | | | | | |
| Soybean VAE | VAE, precultured on EJW1 | Dicot | EJW1 o/n at 28 C. 16/8 | 15 | 1 | 0 | 0.0% | n/a |
| 2/25-3 | o/n at 15 C. dark | Bomb 20 | photoperiod | | | | | |
| Soybean VAE | VAE precultured on EJW1 | Dicot | EJW1 with 8 g/L agarose I | 15 | 1 | 1 | 6.7% | 0% |
| 2/25-4 | with 8 g/L agarose I o/n at | Bomb 20 | o/n at 28 C. 16/8 | | | | | |
| | 15 C. dark | | photoperiod | | | | | |

TABLE 12
Transformation metrics for particle-mediated transformation with DICOTBOMB20 (At_dTP aadA1a) with 2X concentrated bead prep

| | | | | Initial Explants | | | | |
|---|---|---|---|---|---|---|---|---|
| | | | | on 150 ppm | Shoots | T0 | | % |
| Experiment | Preculture conditions | Construct | Rest conditions | spec B5 | harvested | plants | TF | Germline |
| Soybean | VAE, precultured on | Dicot Bomb 20 “2X” | EJW1 o/n at 28 C. 16/8 | 35 | 1 | 1 | 2.9% | 100% |
| VAE 2/25-5 | EJW1 o/n at 15 C. dark | (pellet resuspended in | photoperiod | | | | | |
| | | 18 uL EtOH) | | | | | | |
| Soybean | VAE, precultured on | Dicot Bomb 20 “2X” | EJW1 with 8 g/L | 34 | 1 | 1 | 2.9% | 100% |
| VAE 2/25-6 | EJW1 with 8 g/L agarose | (pellet resuspended in | agarose I o/n at 28 C. | | | | | |
| | I o/n at 15 C. dark | 18 uL EtOH) | 16/8 photoperiod | | | | | |

TABLE 13
GUS segregation data in T1 seedlings produced via particle bombardment
using DICOTBOMB20 (At_dTP aadA1a) or DICOTBOMB 13 (cTP aadA1a)

| | | | | | | | | | Probability |
|---|---|---|---|---|---|---|---|---|---|
| | | Germ- | | | Observed | Observed | Expected | Expected | of chi |
| Experiment | Line | plasm | Bomb | Germline | T1 GUS+ | T1 GUS− | T1 GUS+ | T1 GUS− | square value |
| Soybean VAE 3/17-3 | WP1521-1a | SSW82 | Dicot Bomb 20 | POS | 1 | 10 | 8.25 | 2.75 | 0.0000 |
| Soybean VAE 3/17-3 | WP1521-2a | SSW82 | Dicot Bomb 20 | POS | 1 | 1 | 1.5 | 0.5 | 0.4142 |
| Soybean VAE 3/17-3 | WP1521-8a | SSW82 | Dicot Bomb 20 | neg | 0 | 14 | 10.5 | 3.5 | 0.0000 |
| Soybean VAE 3/17-3B | WP1521-9a | SSW82 | Dicot Bomb 20 | POS | 13 | 1 | 10.5 | 3.5 | 0.1228 |
| Soybean VAE 3/17-4 | WP1521-5a | SSW82 | Dicot Bomb 20 | POS | 6 | 6 | 9 | 3 | 0.0455 |
| Soybean VAE 3/17-4 | WP1521-10a | SSW82 | Dicot Bomb 20 | POS | 5 | 9 | 10.5 | 3.5 | 0.0007 |
| Soybean VAE 3/17-4 | WP1521-13a | SSW82 | Dicot Bomb 20 | no T1 seed | n/a | n/a | n/a | n/a | n/a |
| | | | | set | | | | | |
| Soybean VAE 2/25-1 | WP1520-3a | SSW82 | Dicot Bomb 13 | no T1 seed | n/a | n/a | n/a | n/a | n/a |
| | | | | set | | | | | |
| Soybean VAE 2/25-2 | WP1520-4a | SSW82 | Dicot Bomb 13 | neg | 0 | 11 | 8.25 | 2.75 | 0.0000 |
| Soybean VAE 2/25-2 | WP1520-5a | SSW82 | Dicot Bomb 13 | POS | 7 | 2 | 6.75 | 2.25 | 0.8474 |
| Soybean VAE 2/25-4 | WP1521-6a | SSW82 | Dicot Bomb 20 | neg | 0 | 16 | 12 | 4 | 0.0000 |
| Soybean VAE 2/25-5 | WP1521-3a | SSW82 | Dicot Bomb 20 | POS | 12 | 1 | 9.75 | 3.25 | 0.1495 |
| | | | (2X) | | | | | | |
| Soybean VAE 2/25-6 | WP1521-7a | SSW82 | Dicot Bomb 20 | POS | 11 | 5 | 12 | 4 | 0.5637 |
| | | | (2X) | | | | | | |

### Example 2—Alternative Transit Peptides in Soybean (Glycine max, L.) Transformation with Hra/Imazapyr Selection

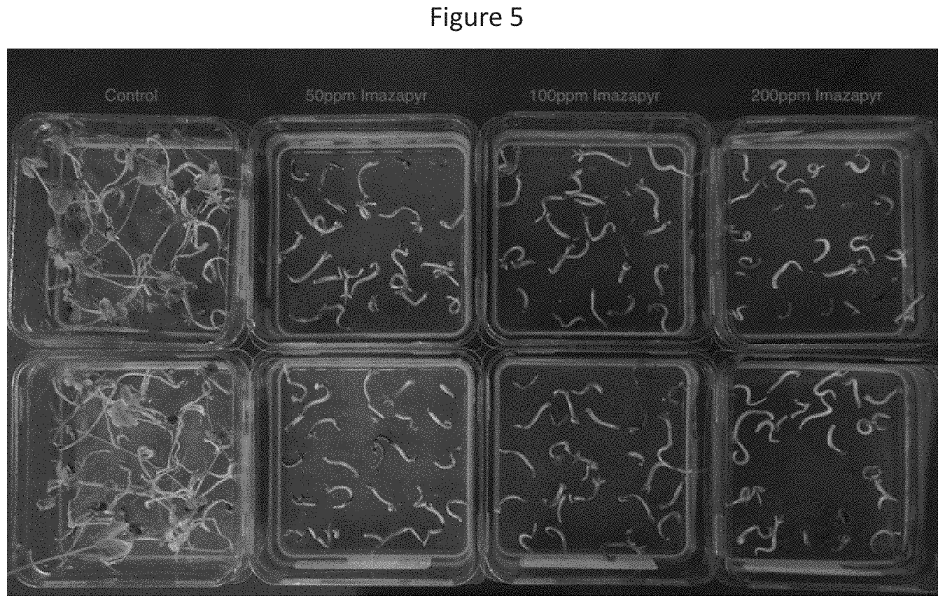

Successful transformation using imazapyr selection has been reported in soybean meristems using particle bombardment (Theon Appl Genet (2000) 101:1-6). We had previously run a selection kill curve on non-transformed soybean VAEs that indicated that imazapyr could be used as a relatively efficient selection agent. Thus, we tested whether we could transform our re-dried soybean VAE meristem explants with Agrobacterium using imazapyr selection with the alternative transit peptide At_dTP. In a pilot study, we tested a large range of imazapyr concentrations, and we found that it was advantageous to use lower concentrations of this herbicide (FIG. 5). Table 14 presents a summary of the data generated in the pilot study, and Table 15 presents the data generated in a follow-up study using lower imazapyr concentrations. Consistent with the aadA1a/spectinomycin selection experiments described in Example 1, only the At_dTP targeting peptide enhances hra/imazapyr selection.

TABLE 14
Transformation metrics for the pilot study

| | Subcellular | | | | | |
|---|---|---|---|---|---|---|
| | location of | Imazapyr Selection | Regenerated | RFP+ | T0 plants | |
| Binary | HRA | Levels (mg/L) | Explants | shoots | to GH | Putative TF |
| DB28 | cytosol | 0.2-20 | 887 | 0 | 0 | 0 |
| DB29 | Ph_cTP | | 878 | 0 | 0 | 0 |
| | (chlor) | | | | | |
| DB30 | At_dTP (chlor + | | 867 | 10 | 7 | 0.8% |
| | mito) | | | | | |

TABLE 15
Transformation metrics for the follow-up study

| | Subcellular | | | | | |
|---|---|---|---|---|---|---|
| | location of | Imazapyr Selection | Regenerated | RFP+ | T0 plants | |
| Binary | HRA | Levels (mg/L) | Explants | shoots | to GH | Putative TF |
| DB28 | cytosol | 1-2.5 | 257 | 0 | 0 | 0 |
| DB29 | Ph_cTP | | 269 | 0 | 0 | 0 |
| | (chlor) | | | | | |
| DB30 | At_dTP (chlor + | | 270 | 8 | 6 | 2.2% |
| | mito) | | | | | |

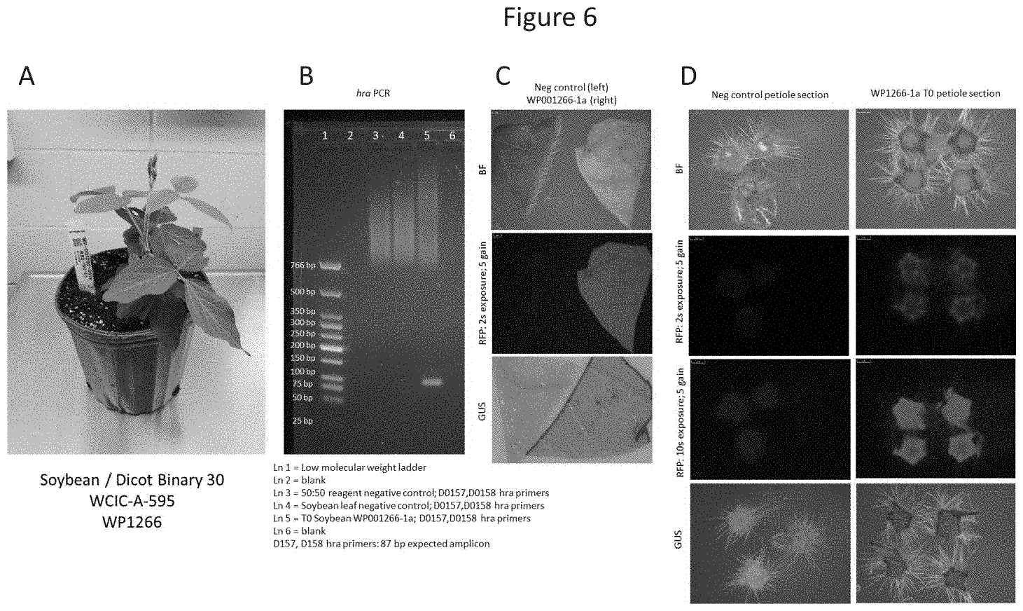

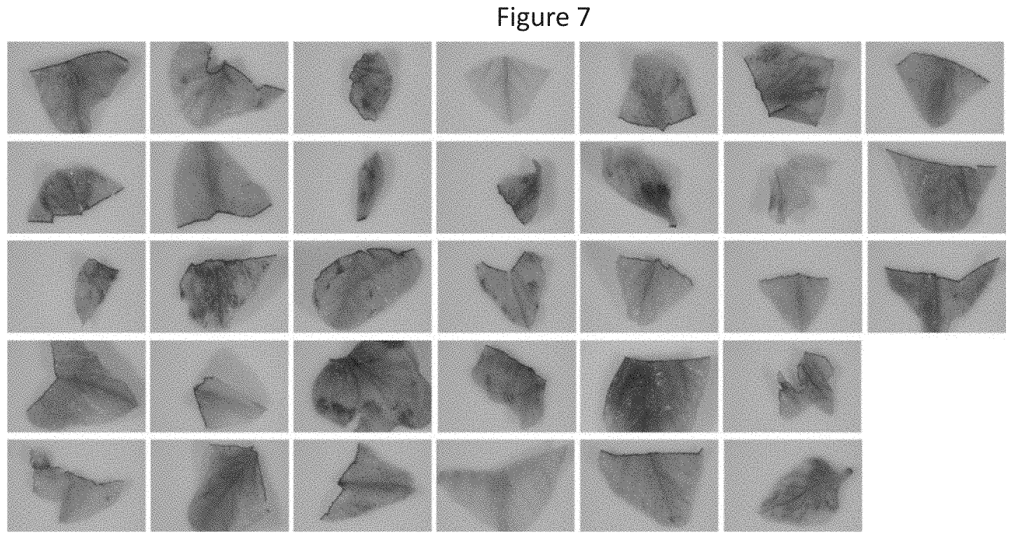

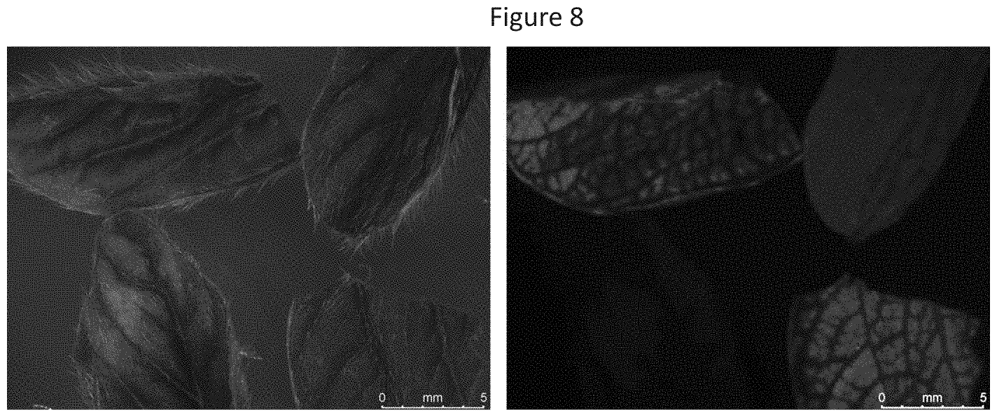

For the first T0 plant we recovered from transforming soybean VAE with imazapyr selection, 5 mg/L imazapyr was used during regeneration. We designated this plant WP1266-1a. WP1266-1a has tested positive for hra by PCR and is both tdTomato-ER-positive and GUS-positive in leaf samples and in vascular bundles of a petiole section, which indicates germline transmission (FIG. 6). We confirmed germline transmission of this event by germinating several T1 seeds and examining T1 leaves for both GUS and tdTomato-ER expression (FIG. 7, FIG. 8).

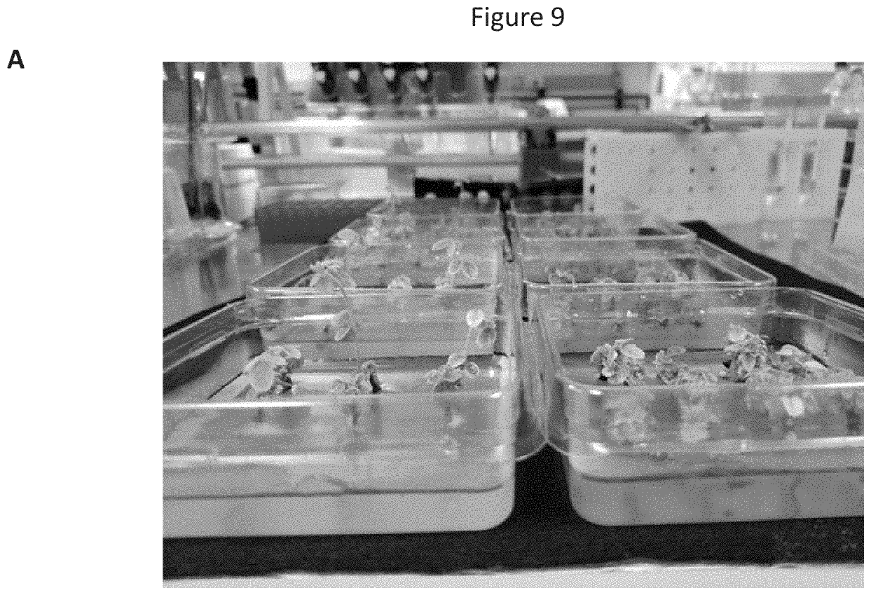

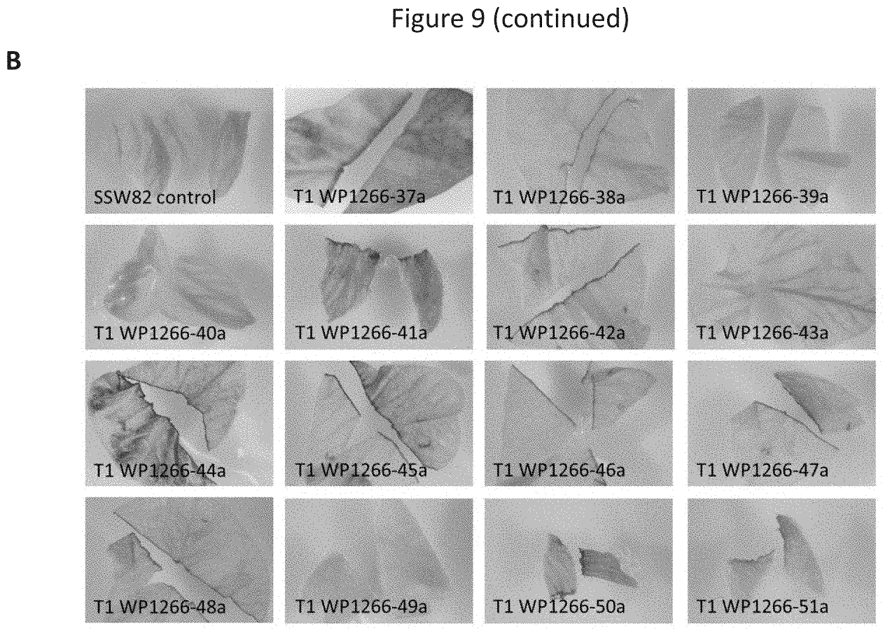

In ongoing work, we are optimizing the imazapyr selection protocol using tdTomato-ER to allow for shoot elongation in positive shoots and to suppress shooting in negative explants. The following protocol appears to promote shooting in tdTomato-ER-positive explants (FIG. 9A) and rooting in the presence of imazapyr appears to be an effective screen for selecting germline transformants.
1 ppm imazapyr B5 for 3-4 weeks
1 ppm imazapyr B5 for 3-4 weeks
0.5 ppm imazapyr B5 for 2-4 weeks
Rooting on 0.025 ppm imazapyr BRM

We tested this optimized imazapyr selection protocol, with and without TDZ, in co-culture using Dicot Binary 30 (At_dTP hra). The results are provided in Table 16. Twelve T1 seed from each line generated from this lead imazapyr protocol were surface sanitized in 20% Clorox for 5 minutes, rinsed, then plated on B5 media. T1 leaves were assayed for GUS expression to determine germline status (Table 17, FIG. 9B). Events WP1266-37 through 44, and WP1266-48, -50, and -51 were generated from the 0 ppm TDZ co-culture treatment, while events WP1266-45 through 47, and WP1266-49 were generated from the 1 ppm TDZ co-culture treatment.

TABLE 16
Transformation and T1 metrics for generated using the optimized
imazapyr selection protocol and DICOTBINARY30 (At_dTP hra)

| | | Explants | | | |
|---|---|---|---|---|---|
| | | inoculated | T0 plants | | |
| | | w/optimized | to GH with | | % |
| Treatment | Binary | protocol | GUS+ roots | TF | Germline |
| 0 ppm TDZ | DICOT | 250 | 11 | 4.4% | 73% |
| in co-culture | BINARY | | | | |
| | 30 | | | | |
| 1 ppm TDZ | DICOT | 225 | 4 | 1.8% | 75% |
| in co-culture | BINARY | | | | |
| | 30 | | | | |

TABLE 17
GUS segregation data for T1 seedlings produced using the optimized imazapyr selection protocol

| | | | | | Observed | Observed | Expected | Expected | Probability |
|---|---|---|---|---|---|---|---|---|---|
| | | | | | T1 | T1 | T1 | T1 | of chi |
| Line | Germplasm | Strain | Binary | Germline | GUS+ | GUS− | GUS+ | GUS− | square value |
| WP1266-37 | SSW82 | Ar18r12v | Dicot Binary 30 | POS | 5 | 2 | 5.25 | 1.8125 | 0.8596 |
| WP1266-38 | SSW82 | Ar18r12v | Dicot Binary 30 | POS | 12 | 0 | 9 | 2.25 | 0.0714 |
| WP1266-39 | SSW82 | Ar18r12v | Dicot Binary 30 | neg | 0 | 9 | 6.75 | 3.9375 | 0.0003 |
| WP1266-40 | SSW82 | Ar18r12v | Dicot Binary 30 | neg | 0 | 7 | 5.25 | 3.0625 | 0.0013 |
| WP1266-41 | SSW82 | Ar18r12v | Dicot Binary 30 | POS | 9 | 3 | 9 | 3 | 1.0000 |
| WP1266-42 | SSW82 | Ar18r12v | Dicot Binary 30 | POS | 5 | 3 | 6 | 2.25 | 0.5186 |
| WP1266-43 | SSW82 | Ar18r12v | Dicot Binary 30 | neg | 0 | 8 | 6 | 3.5 | 0.0006 |
| WP1266-44 | SSW82 | Ar18r12v | Dicot Binary 30 | POS | 9 | 1 | 7.5 | 2.125 | 0.3440 |
| WP1266-48 | SSW82 | Ar18r12v | Dicot Binary 30 | POS | 9 | 3 | 9 | 3 | 1.0000 |
| WP1266-50 | SSW82 | Ar18r12v | Dicot Binary 30 | POS | 8 | 4 | 9 | 3.25 | 0.5940 |
| WP1266-51 | SSW82 | Ar18r12v | Dicot Binary 30 | POS | 8 | 1 | 6.75 | 1.9375 | 0.4078 |
| WP1266-45 | SSW82 | Ar18r12v | Dicot Binary 30 | POS | 10 | 2 | 9 | 2.75 | 0.5742 |
| WP1266-46 | SSW82 | Ar18r12v | Dicot Binary 30 | POS | 8 | 3 | 8.25 | 2.8125 | 0.8873 |
| WP1266-47 | SSW82 | Ar18r12v | Dicot Binary 30 | POS | 7 | 2 | 6.75 | 2.1875 | 0.8735 |
| WP1266-49 | SSW82 | Ar18r12v | Dicot Binary 30 | neg | 0 | 12 | 9 | 5.25 | 0.0000 |

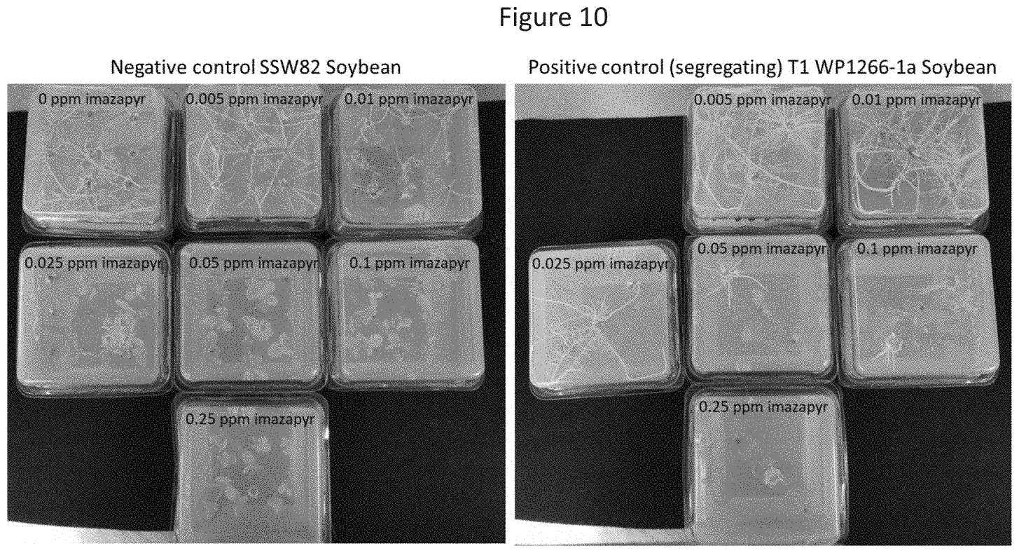

Ongoing experiments also include screening shoots by rooting them in the presence of selection to avoid selecting L1 chimeras/plants transformed in only epidermal layers that will not give rise to T1 positive seed. We are using the confirmed germline event WP1266-1a as a positive control in this effort (FIG. 10). Based on the negative control results, concentrations of imazapyr less than or equal to 0.01 ppm appeared to be insufficient to suppress rooting in non-transformed soybean, while in the positive control we were able to obtain rooting in the 0.025-0.1 ppm imazapyr range. To account for segregation, we assayed leaves from these plants to ensure that rooting events were GUS-positive and that non-rooting plants consisted of nulls. We found a good correlation between robust rooting and GUS expression (Table 18).

TABLE 18
Rooting in presence of various concentrations of imazapyr and
leaf GUS expression in segregating T1 progeny of WP1266-1a

| Imazapyr | | | | |
|---|---|---|---|---|
| concentration | | | | |
| in rooting media | | Plant | Presence of roots | Leaf GUS expression |
| 0.025 | ppm | A | very small roots | GUS negative |
| 0.025 | ppm | B | robust roots | GUS positive |
| 0.025 | ppm | C | no roots | GUS negative |
| 0.05 | ppm | D | no root | GUS negative |
| 0.05 | ppm | E | no roots | GUS negative |
| 0.05 | ppm | F | robust roots | GUS positive |
| 0.1 | ppm | G | robust roots | GUS positive |
| 0.1 | ppm | H | no roots | GUS negative |
| 0.1 | ppm | I | robust roots | GUS positive |

The DICOTBINARY30 construct comprises the At_dTP targeting peptide fused to tdTomato-ER. Thus, using the confirmed germline event WP1266-1a, we should be able to image tdTomato-ER to confirm its expression in the mitochondria and chloroplasts using confocal microscopy.

### Example 3—Alternative Transit Peptides in Cowpea (Vigna unguiculata, L.) Transformation with aadA1a/Spectinomycin Selection

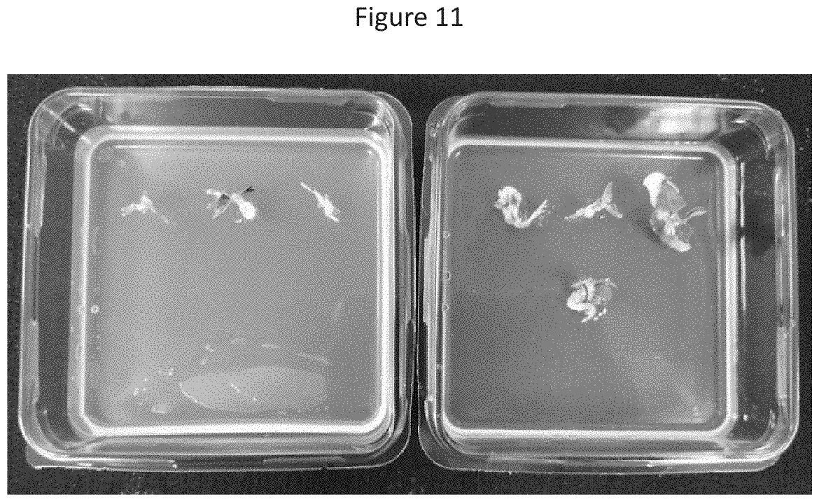

We ran a single experiment comparing the ability of DICOTBINARY19 (cTP aadA) and DICOTBINARY22 (At_dTP aadA) to increase transformation efficiencies in cowpea meristem explants of the variety IT86-1010D. Cowpea transformation was performed as described in US Patent Publication No. US2019/0211347, which is hereby incorporated by reference in its entirety. We used the following protocol for cowpea meristem transformation:
Co-culture for 5 days with 1.75 ml INO with 1 ppm TDZ per plantcon
Embed explants (orienting apical meristems upward and radicals in media) directly after co-culture
Select using 150 mg/L spectinomycin (corrected for activity) post co-culture for 3-4 weeks
Transfer phenotype positive explants to 50 mg/L spectinomycin (active) for 3-4 weeks and then to non-selective B5
   The results of this experiment are consistent with the soybean results. They demonstrate that At_dTP results in greater transformation frequency in cowpea as compared to cTP (Table 19, FIG. 11).

TABLE 19
Transformation metrics for the cowpea study

| | | | | | | Greening | | | |
|---|---|---|---|---|---|---|---|---|---|
| | | | | | Explants | explants | | | |
| | | | | | to 150 | transferred | | | |
| | | | | | ppi spec | to 50 ppm | Greening | T0 | |
| | | | | Subcellular | B5 after | spec B5 | explants | plants | |
| | | | | location of | co- | selection | transferred | to | |
| Species | Genotype/Line | Strain | Binary | aadA1a | culture | media | to B5 | GH | TF |
| Cowpea | IT86D-1010 | Ar18r12v | DB19 | cTP | 145 | 3 | 1 | 0 | 0.0% |
| Cowpea | IT86D-1010 | Ar18r12v | DB22 | At_dTP | 155 | 4 | 3 | 2 | 1.3% |

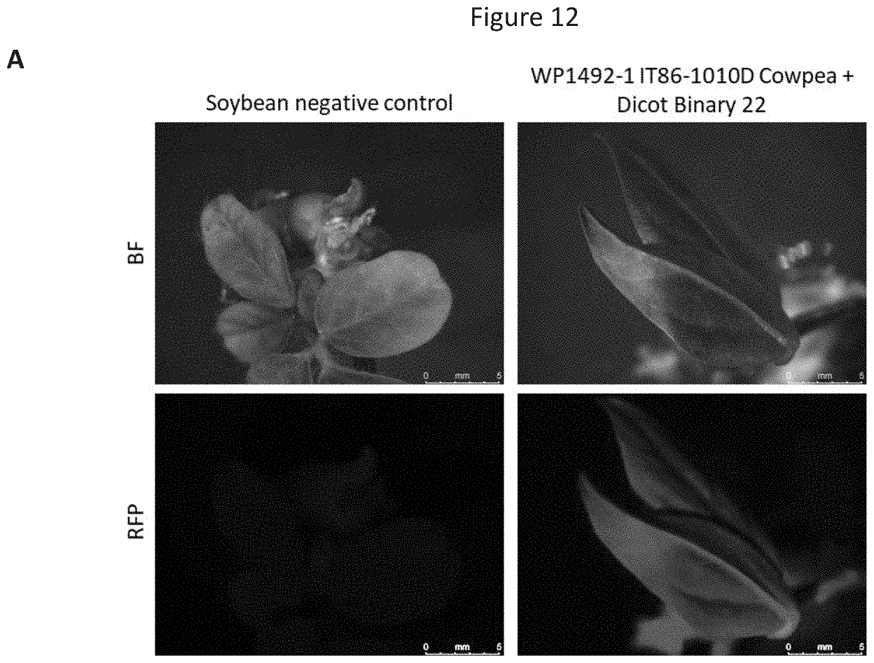

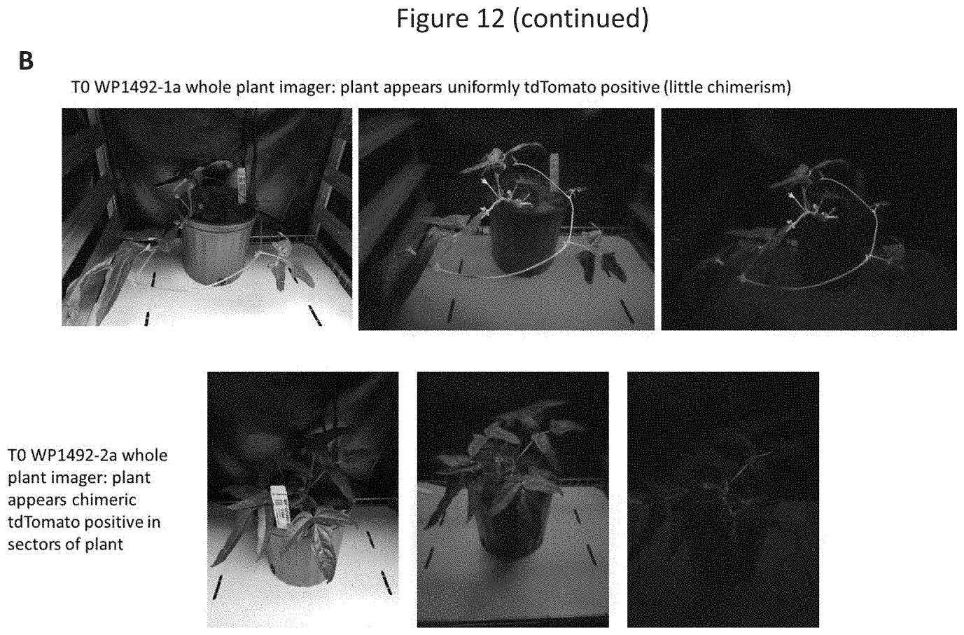

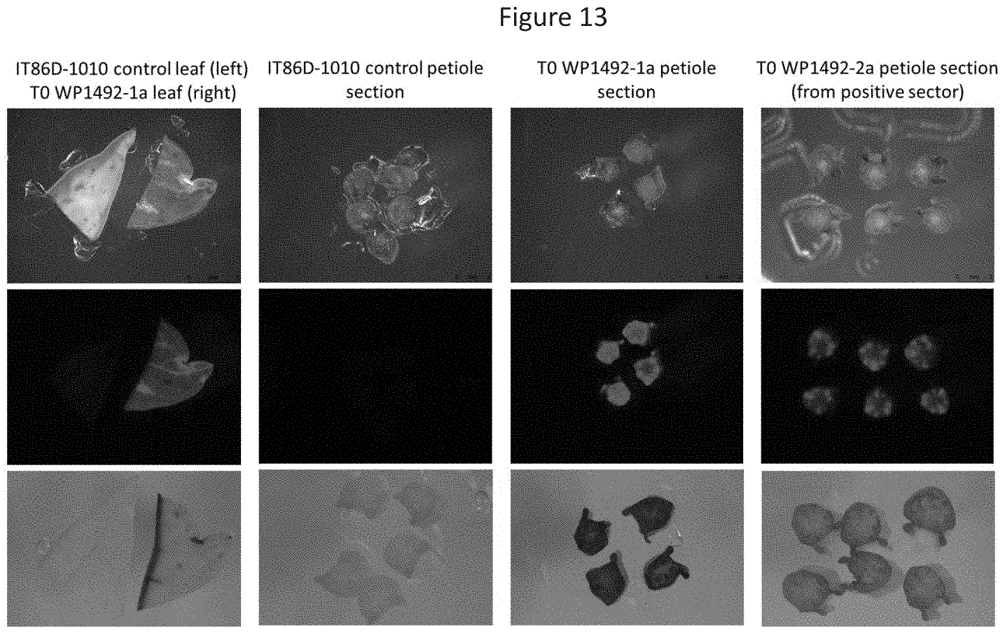

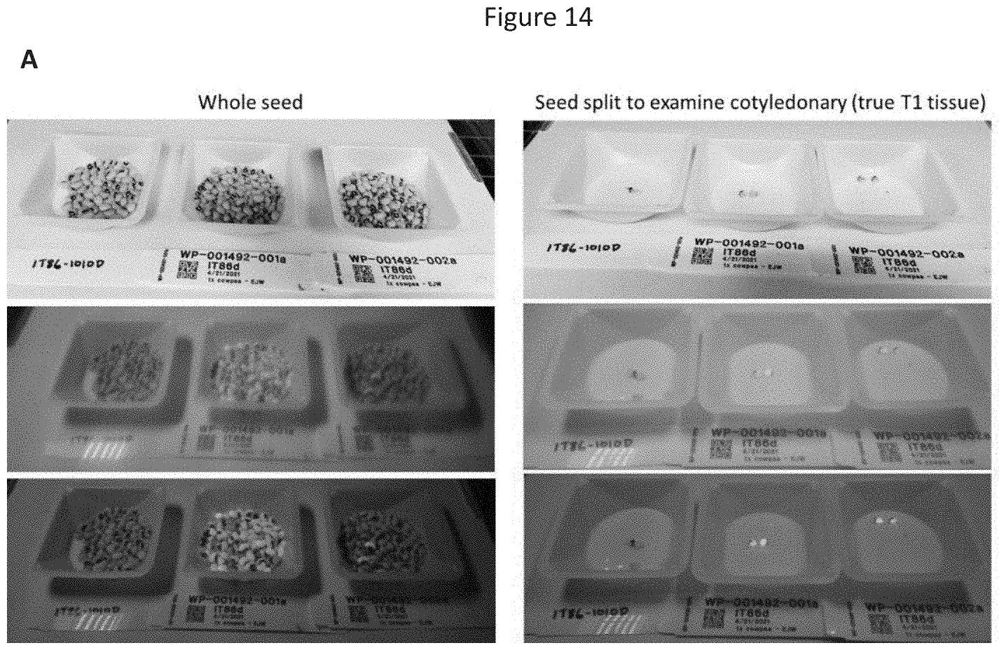

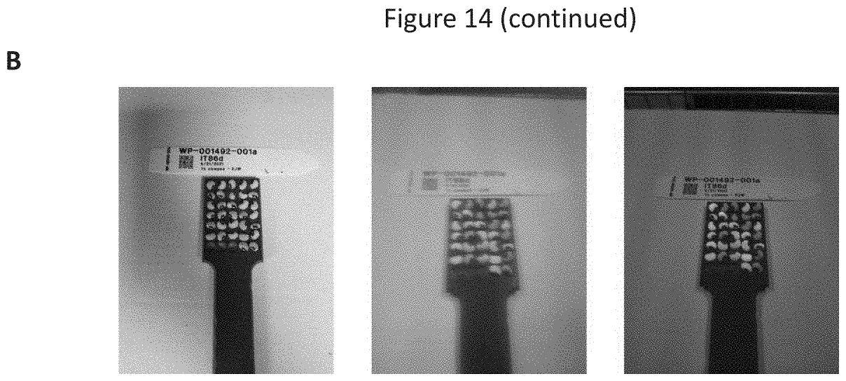

We imaged tdTomato-ER expression in the T0 cowpea events generated using DICOTBINARY22 (At_dTP aadA) (FIG. 12). Both identified events, i.e., WP1492-1a and WP1492-2a, were tdTomato-ER positive. However, WP1492-1a showed more uniform expression and WP1492-2a showed chimeric expression. We also took petiole sections of these events and found that both tdTomato-ER and GUS are expressed in the vascular bundles (FIG. 13), which is an early indicator of germline status. This result was confirmed by tdTomato-ER expression in T1 cowpea seeds (FIG. 14, Table 20). These data demonstrate germline transformation of cowpea meristems using At_dTP aadA.

TABLE 20
tdTomato-ER segregation data in T1 seedlings produced using DICOTBINARY22 (At_dTP aadA)

| | | | | | | | | | Probability |
|---|---|---|---|---|---|---|---|---|---|
| | | | | | Observed T1 | Observed T1 | Expected T1 | Expected T1 | of chi |
| Line | Germplasm | Strain | Binary | Germline | aadA+ | aadA− | aadA+ | aadA− | square value |
| WP1492-1 | IT86D- | Ar18r12v | Dicot | POS | 20 | 12 | 24 | 8 | 0.1025 |
| | 1010 | | Binary 22 | | | | | | |
| WP1492-2 | IT86D- | Ar18r12v | Dicot | POS* | 0 | 32 | 24 | 8 | 0.0000 |
| | 1010 | | Binary 22 | | | | | | |

*2 T1 seeds identified positive for tdTomato-ER

### Example 4—Alternative Transit Peptides in Hemp (Cannabis sativa, L.) Transformation with aadA1a/Spectinomycin Selection

We ran an experiment using Cannabis meristem explants of the variety Badger inoculated with Ar18r12v/DICOTBINARY22 (At_dTP aadA1a). Cannabis transformation was performed as described in US Patent Publication No. US2021/0071186, which is hereby incorporated by reference in its entirety. We used the following transformation protocol:
Imbibe seeds at 37° C. in bean germinating medium in the dark
Co-culture for 5 days in the dark with 1.25-1.5 ml INO with 1 ppm TDZ per plantcon
Select using 50 ppm spectinomycin (active) hemp node medium with 0.5 ppm meta-topolin
Root on WPM-based bean rooting media (WP1331-1a) or hemp rooting media (WP1331-2a)
   The media used in this protocol are detailed in Tables 21-24, below.

TABLE 21
Bean germinating medium composition

| | Amount to add per liter |
|---|---|
| Ingredients and Notes | (grams) |
| Phytotechnology Laboratories WPM L499 | 2.41 |
| Sucrose | 20 |
| pH to 5.8 with 1N KOH and autoclave | |
| Add the following prior to use: | |
| Captan fungicide (50WP) | 0.06 |
| Bravo fungicide (Daconil) (82DP) | 0.03 |
| Cefotaxime (100 mg/ml) | 1.25 ml |

TABLE 22
Hemp node medium composition

| | Amount to add per liter |
|---|---|
| Ingredients and Notes | (grams) |
| MS Salts complete with vitamins | 4.43 |
| (PhytoTech M519) | |
| Sucrose | 30 |
| Cleary's 3336 | 0.06 |
| pH to 5.7 with 1N KOH | |
| Agar (Sigma A7921) | 8 |
| Autoclave | |
| Meta-topolin (mT) (1 mg/ml) | 0.5 ml |
| Carbenicillin (100 mg/ml) | 4 ml |
| Cefotaxime (100 mg/ml) | 2 ml |
| Timetin (150 mg/ml) | 1 ml |
| Selection | as needed |

TABLE 23
WPM-based bean rooting medium composition

| | Amount to add per liter |
|---|---|
| Ingredients and Notes | (grams) |
| Phytotechnology Laboratories | 2.41 |
| WPM L499 | |
| Myo-inositol | 0.1 |
| Sucrose | 30 |
| pH 5.8 with KOH | |
| Agar, Sigma A7921 | 8 |
| Autoclave | |
| Add after autoclaving: | |
| Cysteine (100 mg/ml) - make | Use at 1.0 ml per Liter (100 mg/L) |
| stock fresh | |
| Cefotaxime (100 mg/ml) | Use at 2.0 ml per Liter (200 mg/L) |
| IAA (1 mg/ml) | Use at 0.1 ml per Liter (0.1 mg/L) |
| MS Vitamins (1000X) | Use at 1.0 ml per Liter |
| Selection | as needed |

TABLE 24
Hemp rooting medium composition

| | Amount to add per liter |
|---|---|
| Ingredients and Notes | (grams) |
| MS Salts complete with vitamins | 2.215 |
| (PhytoTech M519) | |
| Sucrose | 15 |
| pH to 5.6 with KOH | |
| Agar (Sigma A7921) | 8 |
| Autoclave | |
| IBA stock (1 mg/ml - Ed has stock) | 0.51 ml = 510 uL |
| Carbenicillin (100 mg/ml stock) | 2.5 ml |
| Cefotaxime (100 mg/ml stock) | 2 ml |
| Timentin (150 mg/ml stock) | 1 ml |
| Selection | as needed |

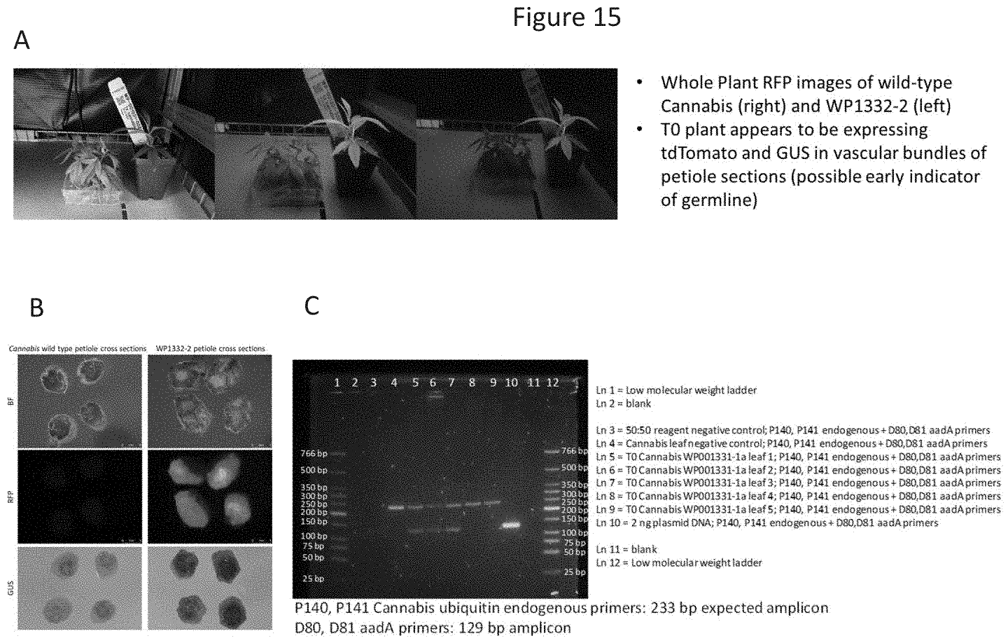

47 explants were inoculated with 1.25 ml of the co-culture treatment, and this produced 1 T0 plant, WP1331-2a (TF 2.1%). 55 explants were inoculated with 1.5 ml of the co-culture treatment, and this also produced 1 T0 plant, WP1331-1a (TF 1.8%). The WP1331-1a T0 plant tested positive for aadA1a by PCR in ⅗ leaves. The WP1331-2a T0 plant expressed tdTomato-ER throughout the plant and was both tdTomato-ER-positive and GUS-positive in the vascular bundles of a petiole section (FIG. 15).

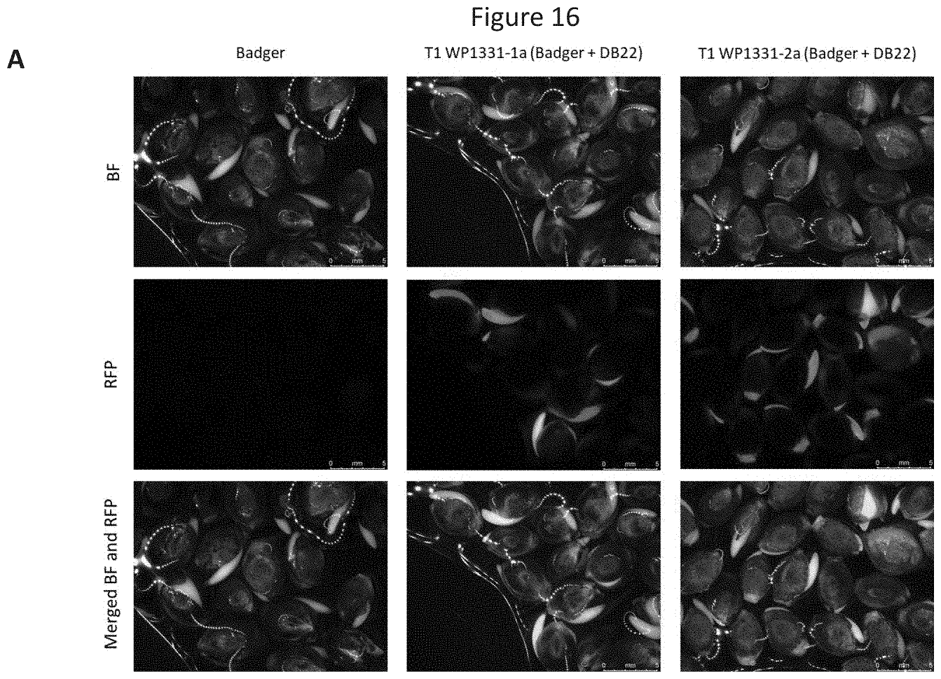

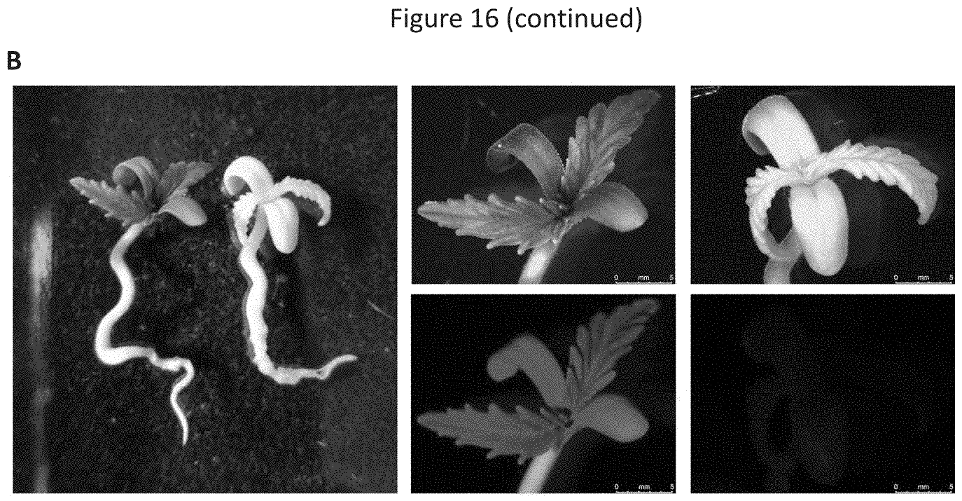

We confirmed germline transmission of these events (FIG. 16A). We then germinated the T1 seeds of these events on a liquid formulation of Hemp Node Media containing 25 mg/L spectinomycin to determine segregation frequencies (Table 25). Within 2 weeks, null T1 seedlings were bleached while transgenic T1 seedlings remained greening and expressing tdTomato-ER (FIG. 16B).

TABLE 25
Segregation data for T1 seedlings of Cannabis lines produced using DICOTBINARY22 (At_dTP aadA1a)

| | | | | | | | | | Probability |
|---|---|---|---|---|---|---|---|---|---|
| | | | | | Observed T1 | Observed T1 | Expected T1 | Expected T1 | of chi |
| Line | Germplasm | Strain | Binary | Germline | aadA+ | aadA− | aadA+ | aadA− | square value |
| WP1331-1 | Badger | Ar18r12v | Dicot | POS | 3 | 8 | 5.5 | 5.5 | 0.1317 |
| | | | Binary 22 | | | | | | |
| WP1331-2 | Badger | Ar18r12v | Dicot | POS | 9 | 6 | 7.5 | 7.5 | 0.4386 |
| | | | Binary 22 | | | | | | |

### Example 5—Alternative Transit Peptides in Chickpea (Cicer arietinum, L.) Transformation with aadA1a/Spectinomycin Selection

We ran experiments on chickpea meristem explants with DICOTBINARY19 (cTP aadA1a), DICOTBINARY21 (ER aadA1a), and DICOTBINARY22 (At_dTP aadA1a) using the following transformation protocol:
Remove shoot apical meristem (SAM) after excision prior to inoculation and co-culture
Co-culture for 5 days with 2.5 ml INO with 1 ppm TDZ per plantcon
Embed explants (orienting apical meristems upward and radicals in media) directly after co-culture and for subsequent transfers for greening, phenotype positive explants on spectinomycin
Select using 150 mg/L streptomycin post co-culture for 3-4 weeks
Transfer phenotype positive explants to 50 mg/L spectinomycin (active) for 3-4 weeks and then to non-selective B5 medium for 2 weeks
Harvest shoots onto 25 mg/L spectinomycin WPM-based rooting media at each stage if sufficiently elongated

Notably, we included ER-targeted aadA1a (DICOTBINARY21) in this study even though it did not increase transformation efficiency in soybean because the endoplasmic reticulum is associated with plastids in plant cells, and we hypothesized that placement of the spectinomycin degrading activity within the ER might protect to the associated plastids.

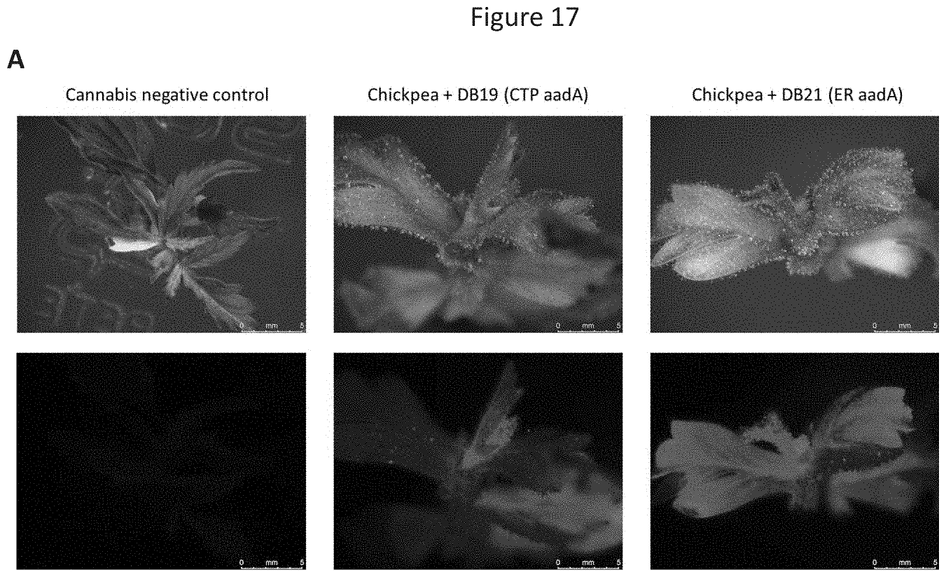

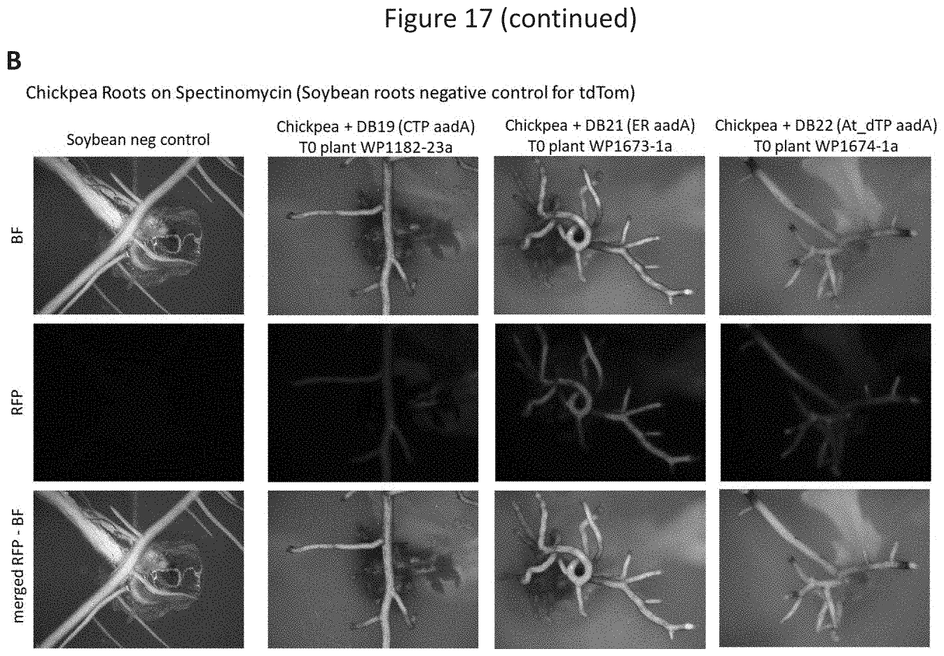

We obtained T0 plants that express tdTomato-ER in both leaves and roots that were generated using DICOTBINARY19, DICOTBINARY21, and DICOTBINARY22 and selected on 25 mg/L spectinomycin (FIG. 17B,C; Table 26). Notably, the T0 plants obtained with DICOTBINARY22 were derived from later tests. These later tests involved varying the amount of TDZ used in the co-culture (Table 27) and using PEG as an osmotic agent when the chickpea shoot apical meristem (SAM) is removed prior to inoculation (Table 28). The expression of tdTomato-ER in chickpea roots is an early indicator of germline transformation using these three binaries.

TABLE 26
Transformation metrics for the initial chickpea experiment. 50 mg/L spectinomycin was
used for the second selection and 25 mg/L spectinomycin was used in the rooting media.

| | | | RFP | | | |
|---|---|---|---|---|---|---|
| | Subcellular localization | | positive | Shooting | T0 | Transformation |
| Binary | of aadA | Explants | shoots | frequency (SF) | plants | frequency (TF) |
| Dicot | Chloroplast (cTP) | 457 | 15 | 3.3% | 6 | 1.3% |
| Binary 19 | | | | | | |
| Dicot | Endoplasmic reticulum | 358 | 8 | 2.2% | 2 | 0.6% |
| Binary 21 | (ER) | | | | | |
| Dicot | Mitochondria and | 469 | 21 | 4.5% | 0 | 0.0% |
| Binary 22 | Chloroplast (At_dTP) | | | | | |

TABLE 27
Transformation metrics for chickpea experiment in which
the amount of TDZ used in co-culture was varied

| | | Co-culture | | | Shooting | | |
|---|---|---|---|---|---|---|---|
| Strain | Binary | conditions | Explants | Shoots | frequency (SF) | T0 plants | TF |
| Ar18r12v | Dicot Binary 22 | 0 ppm TDZ | 183 | 0 | 0.0% | 0 | 0.0% |
| Ar18r12v | Dicot Binary 22 | 1 ppm TDZ | 168 | 15 | 8.9% | 4 | 2.4% |
| Ar18r12v | Dicot Binary 22 | 0 ppm TDZ | 57 | 0 | 0.0% | 0 | 0.0% |
| Ar18r12v | Dicot Binary 22 | 0.25 ppm | 63 | 8 | 12.7% | 2 | 3.2% |
| | | TDZ | | | | | |
| Ar18r12v | Dicot Binary 22 | 0.5 ppm TDZ | 50 | 3 | 6.0% | 1 | 2.0% |
| Ar18r12v | Dicot Binary 22 | 1 ppm TDZ | 33 | 3 | 9.1% | 0 | 0.0% |

TABLE 28
Transformation metrics for chickpea experiment in which
PEG was added as an osmotic agent after SAM removal

| | | Pre-inoculation | | | Shooting | T0 | |
|---|---|---|---|---|---|---|---|
| Strain | Binary | conditions | Explants | Shoots | frequency (SF) | plants | TF |
| Ar18r12v | Dicot Binary 22 | PEG | 216 | 15 | 6.9% | 3 | 1.4% |
| Ar18r12v | Dicot Binary 22 | H2O | 208 | 11 | 5.3% | 1 | 0.5% |

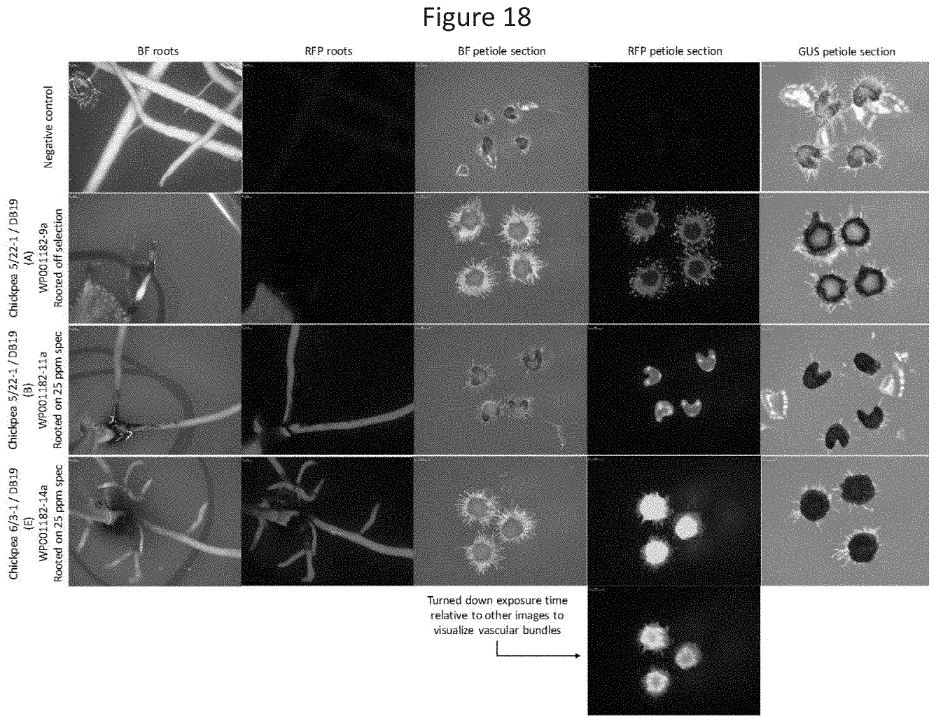

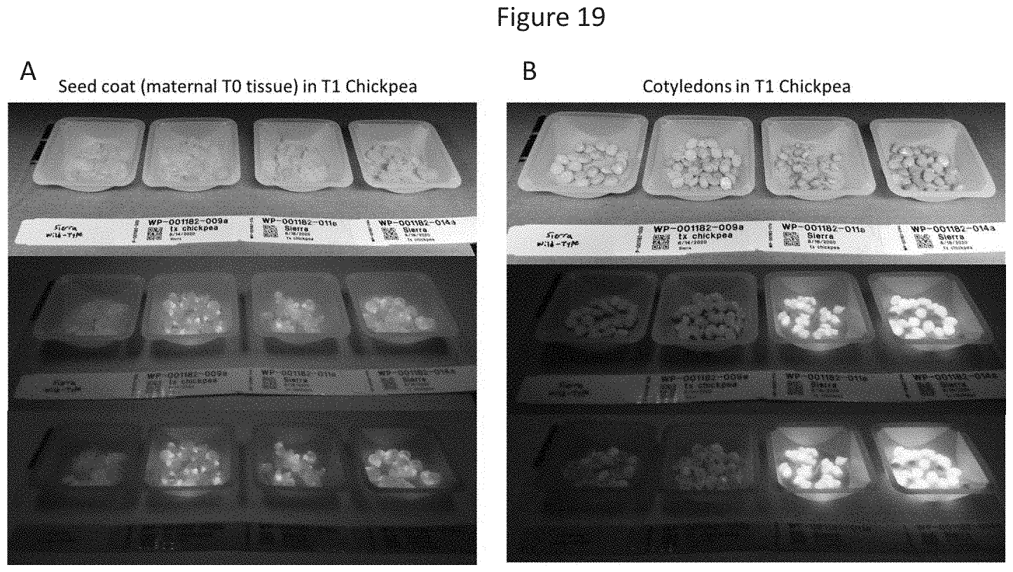

Using the optimized chickpea protocol, we observed an enrichment of T0 plants expressing tdTomato-ER and GUS in roots and in vascular tissue within petiole sections of chickpea (FIG. 18). Expression of tdTomato-ER in roots and in vascular bundles of petiole section appears to be an indicator of germline status, as the T1 seed of WP1182-11a and -14a expressed tdTomato-ER in cotyledons (true T1 tissue) and maternal seed coats (T0 tissue), whereas the T1 seed of WP-1182-9a did not express tdTomato-ER in cotyledons and expression was limited to seed coat T0 tissue (FIG. 19).

### Example 6—Alternative Transit Peptides in Pinto Bean (Phaseolus vulgaris, L.) Transformation with aadA1a/Spectinomycin Selection

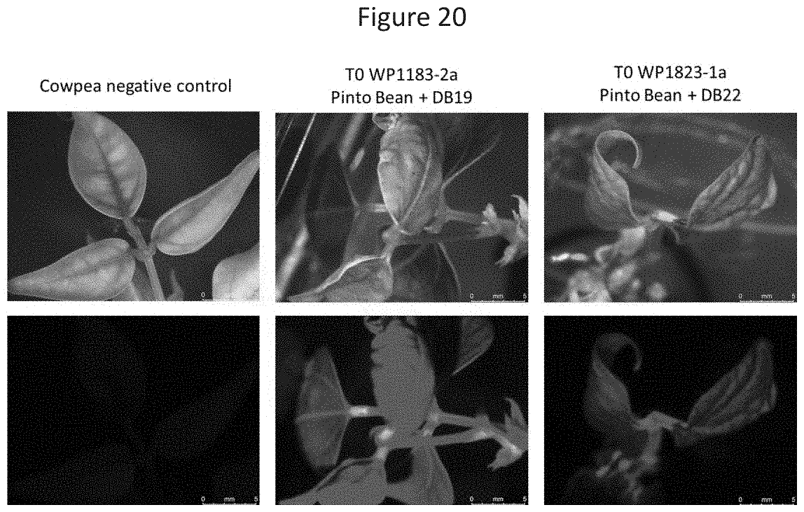

We ran experiments with DICOTBINARY19 (cTP aadA1a) and DICOTBINARY22 (At_dTP aadA1a) in pinto bean using the protocol provided in US Patent Publication No. 2019/0211347, which is hereby incorporated by reference in its entirety. We modified this protocol to add a vacuum infiltration step after the sonication step based on promising transient results in another variety of pinto bean. The results of this experiment are presented in Table 29 and FIG. 20. These results demonstrate that both DICOTBINARY19 (cTP aadA1a) and DICOTBINARY22 (At_dTP aadA1a) were able to produce germline events in pinto bean.

TABLE 29
Transformation metrics for pinto bean experiment

| | | | | | tdTom+ | | |
|---|---|---|---|---|---|---|---|
| | | Explants to 25 mg/L | | | phenotypes to | Plants | Putative |
| Species | Genotype/Line | spectinomycin B5 | Strain | Binary | B5 | to GH | TF |
| Dry | Pinto | 173 | 18r12v | Dicot | 7 | 4 | 2.3% |
| Bean | | | | Binary | | | |
| | | | | 19 | | | |
| Dry | Pinto | 182 | 18r12v | Dicot | 11 | 3 | 1.6% |
| Bean | | | | Binary | | | |
| | | | | 22 | | | |

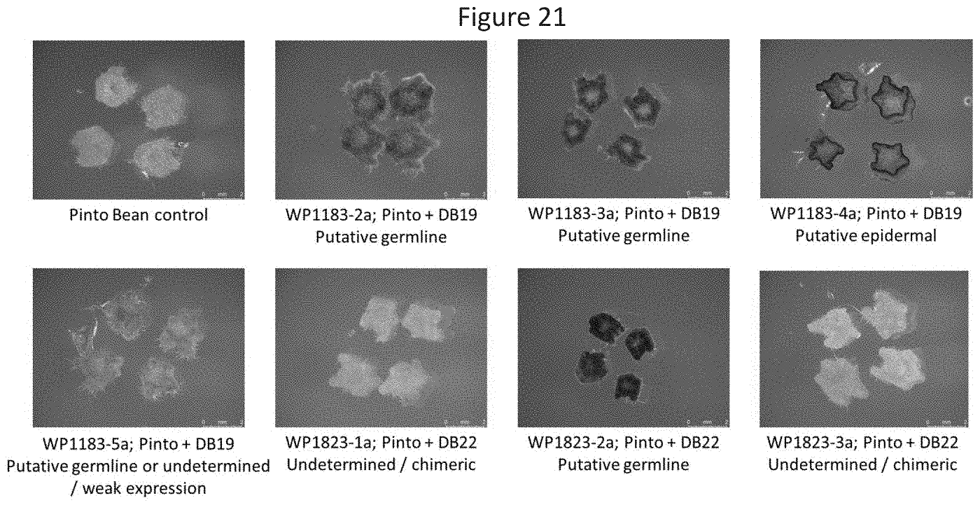

We took petiole sections of the transgenic pinto bean events to determine if we could detect GUS in the vascular bundles, an early indicator of germline transformation in our mature meristem-based system (FIG. 21). The events WP1183-2a (DB19), WP1183-3a (DB19), and WP1823-2a (DB22) showed clear GUS expression in the vascular bundles. Weaker GUS expression, possibly in the vascular bundles, was detected in WP1183-5a (DB19). These events demonstrate that alternate transit peptides can be used to transform pinto bean with aadA. Notably, the event WP1183-4a appears to be epidermal, and WP1823-1a and -3a are likely chimeric, as the sampled petioles did not appear to express GUS.

### Example 7—Alternative Transit Peptides in Pea (Pisum sativum, L.) Transformation with aadA1a/Spectinomycin Selection

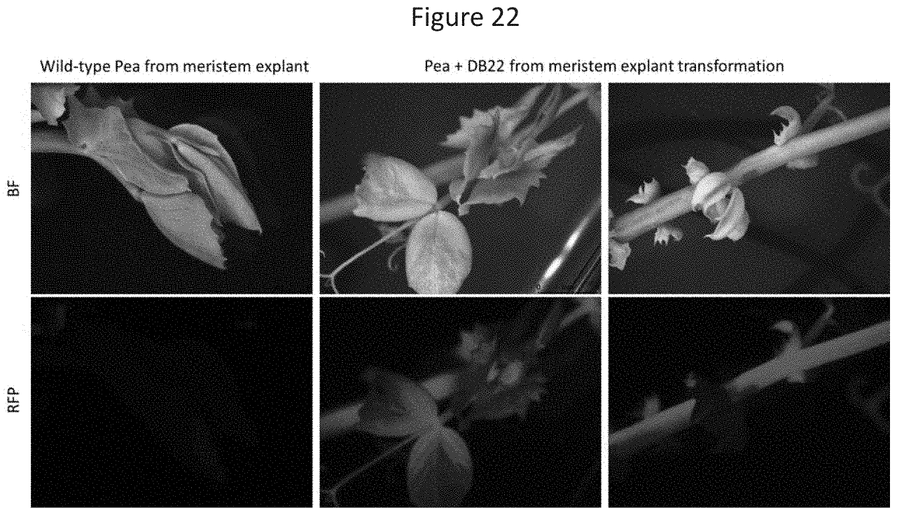

We ran a transformation test with DICOTBINARY22 (At_dTP aadA1a) in pea meristems using a modified chickpea protocol. We recovered a tdTomato-ER positive shoot (FIG. 22). Further tests are ongoing.

### Example 8—Construct Design

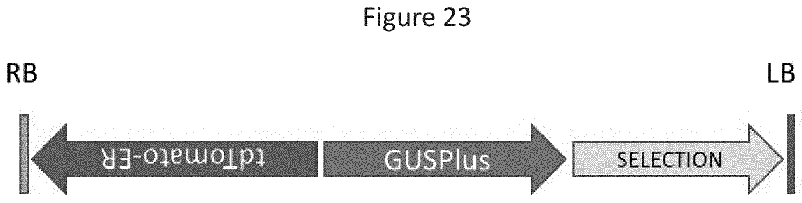

T-DNA generation in Agrobacterium starts with a single stranded nick at the T-DNA right border, followed by covalent attachment of the virD2 protein to the nascent single-stranded T-DNA molecule. The full-length T-DNA is generated with a second nick at the T-DNA left border. The virD2::T-DNA is trafficked from the Agrobacterium cell into the plant cell, where the T-DNA is further trafficked to the plant cell nucleus, where the T-DNA is integrated into the plant genome. In a final genomic sequence resulting from a transgenic event, it is common to lose part of the transgene sequence or have some run-on (i.e., gain extra sequence) from the left gene border (see, e.g., Plant J (2018) doi: 10.1111/tpj.13992). Thus, the inventors have designed their constructs with the plant selection marker gene oriented such that its terminator is proximal to the T-DNA left border, and that transcription of the reporter gene is directed towards the T-DNA left border, as depicted schematically in FIG. 23. With this orientation, even small truncations of the T-DNA at the left border are likely to result in an obvious phenotype (i.e., loss of selection marker gene expression) since the polyadenylation signal in the terminator might be lost. In the opposite scenario in which the promoter is located proximal to the left border, sequence truncations could produce a wide array of reporter gene phenotypes without fully eliminating selection marker gene expression. Further, when a promoter is located at the end of a T-DNA, its activity can be influenced by nearby genomic sequences. These variable results are undesirable in a transgenic plant production facility, which seeks to generate plants that express consistent levels of a reporter gene. By orienting the selection marker such that the terminator is proximal to the T-DNA left border, the chemical selection will eliminate those events in which truncations have rendered the selection marker transcriptional unit less than optimally functional. Pragmatically, this means that the transgenic plants which are produced will be those with robust expression of the selection marker gene. Since T-DNA truncation is mainly a left border phenomenon, one can infer, from robust selection marker gene expression, that the other transcriptional units included in the T-DNA will also be present.

What is claimed:

1. A DNA construct comprising a first expression cassette comprising a first heterologous promoter operably linked to a sequence encoding a transit peptide and a target protein, wherein the transit peptide is at least 90% identical to SEQ ID NO:3 or SEQ ID NO:9.

2. The DNA construct of claim 1, wherein the sequence encoding the transit peptide is translationally fused to the sequence encoding the target protein.

3. The DNA construct of claim 1, wherein the transit peptide is SEQ ID NO:3 or SEQ ID NO:9.

4. The DNA construct of claim 1, wherein the target protein is a selectable marker or a reporter gene.

5. The DNA construct of claim 4, wherein the selectable marker confers resistance to an antibiotic or an herbicide.

6. The DNA construct of claim 5, wherein the selectable marker is encoded by SEQ ID NO:12 or SEQ ID NO:13.

7. The DNA construct of claim 1, further comprising a second expression cassette comprising a second heterologous promoter.

8. The DNA construct of claim 1, wherein the DNA construct comprises SEQ ID NO:5, SEQ ID NO:6, SEQ ID NO:10, or SEQ ID NO:11.

9. A vector comprising the DNA construct of claim 1.

10. The vector of claim 9, wherein the vector is a T-DNA vector for agrobacterium-mediated transformation.

11. The vector of claim 9 formulated for gene gun transformation.

12. A plant cell comprising the DNA construct of claim 1.

13. The plant cell of claim 12, wherein the plant cell expresses the transit peptide and the target protein as a fusion protein.

14. The plant cell of claim 13, wherein the fusion protein is targeted to the mitochondria and the chloroplasts within the plant cell.

15. The plant cell of claim 12, wherein the plant cell is a soybean, cowpea, hemp, chickpea, pinto bean, or pea plant cell.

16. A method of targeting a target protein to the chloroplasts and mitochondria of a plant cell, the method comprising: transforming the plant cell with a construct comprising a heterologous promoter operably linked to a sequence encoding a transit peptide and the target protein, wherein the transit peptide is at least 90% identical to SEQ ID NO:3 or SEQ ID NO:9.

17. A method of producing a transgenic plant, the method comprising:
a) transforming an explant with a construct comprising a heterologous promoter operably linked to a sequence encoding a transit peptide and a selectable marker, wherein the transit peptide is at least 90% identical to SEQ ID NO:3 or SEQ ID NO:9;
b) contacting the explant with a selective agent; and
c) regenerating a transformed plant from the transgenic cells;
wherein the selectable marker confers an ability to grow in the presence of the selective agent.

18. The method of claim 17, wherein the selectable marker is encoded by the aadA1a gene and the selective agent is spectinomycin or wherein the selectable marker is encoded by the hra gene and the selective agent is imazapyr.

19. The method of claim 17, wherein the plant is a dicot.

20. The method of claim 17, wherein the plant is a monocot.

21. The method of claim 17, wherein the construct further comprises a second heterologous promoter operably linked to a sequence that encodes a target protein.
